(19) United States
(12) Patent Application Publication
(10) Pub. No.: US 2012/0322064 A1
(43) Pub. Date: Dec. 20, 2012

(54) HYBRIDIZATION PROBES AND METHODS OF THEIR USE

(75) Inventors: Evangelyn C. Alocilja, East Lansing, MI (US); Sudeshna Pal, Orlando, FL (US)

(73) Assignee: BOARD OF TRUSTEES OF MICHIGAN STATE UNIVERSITY, East Lansing, MI (US)

(21) Appl. No.: 13/477,199

(22) Filed: May 22, 2012

Related U.S. Application Data
(60) Provisional application No. 61/519,468, filed on May 23, 2011.

(51) Int. Cl. G01N 27/26; C12M 1/34; B82Y 15/00

(52) U.S. Cl. 435/6.11; 436/501; 435/287.2; 422/69; 977/924; 977/773

(57) ABSTRACT

Hybridization probes for hybridizing to the same target nucleic acid are disclosed, the hybridization probes comprising an electrically-active magnetic nanoparticle-labeled detector probe and a capture probe including a conjugating moiety for immobilization. Also disclosed is a biodetection method including the steps of: providing hybridization probes for hybridizing to the same target nucleic acid, the hybridization probes comprising an electrically-active magnetic nanoparticle-labeled detector probe and a capture probe; hybridizing the target nucleic acid with each of the electrically-active magnetic nanoparticle-labeled detector probe and a capture probe in a sample including the target nucleic acid; magnetically separating the hybridized target nucleic acid from the sample; capturing the hybridized target nucleic acid on a substrate through the capture probe; and measuring the oxidation-reduction signal of the electrically-active magnetic nanoparticle-labeled detector probe.

### CROSS-REFERENCE TO RELATED APPLICATION

Priority is claimed to U.S. Provisional Application No. 61/519,468, filed May 23, 2011, the entire contents of which is incorporated herein by reference in its entirety.

### STATEMENT OF GOVERNMENT INTEREST

This invention was made with government support from the Department of Homeland Security under grant number DHS-NCFPD Subaward No. X9106025105. The government has certain rights in the invention.

### REFERENCE TO A SEQUENCE LISTING SUBMITTED IN A COMPUTER READABLE FORMAT

The application contains nucleotide sequences which are identified with SEQ ID NOs. The Sequence Listing provided in computer readable form, incorporated herein by reference in its entirety, is identical to the written copy of the Sequence Listing provided with the application.

### BACKGROUND OF THE DISCLOSURE

1. Field of the Disclosure

The disclosure relates to hybridization probes for hybridizing to the same target nucleic acid, where a first hybridization probe includes an electrically-active magnetic nanoparticle-labeled detector probe and a second hybridization probe includes a capture probe with a conjugating or other immobilization moiety.

2. Brief Description of Related Technology

Separating and concentrating targeted-biological materials from complex clinical, environmental, and food sample matrices for diagnostic purposes are often challenging and insufficient to enable detection methods that are rapid and highly sensitive.

Current cell separation systems utilize magnetic bead technology and super-paramagnetic particles to isolate desired cells from a mixture. The magnetic beads are coated with an affinity group specific for a cell's surface antigens. In positive (or direct) isolation, the targeted cells are labeled. A suspension of the coated magnetic beads is mixed with target cells. Following incubation, during which the target cells bind to the ligands on the beads, a magnetic field is applied to the suspension to immobilize the magnetic particles and the target cells. Any unbound material is then flushed from the system. Removing the magnetic field releases the desired cells. In depletion (or indirect) isolation, the unwanted cells are immobilized with the paramagnetic beads, allowing the desired cells to be flushed and then collected when the magnetic field is applied. However, for real applications it is necessary to design detection systems that are able to detect the targets from complex systems as most environmental samples will have interferences from the matrices.

Alocilja et al. U.S. Publication Nos. 2003/0153094, 2008/0314766, 2009/0123939, generally relate to biosensor devices and/or BEAM nanoparticle compositions and are incorporated herein by reference in their entireties.

### SUMMARY

Features such as sensitivity, efficiency, and cost could be enhanced if particles that are used for separation and concentration of targets could be used to directly measure a change in some target-induced physical attribute of the particles.

The present disclosure provides hybridization probes including electrically-active magnetic nanoparticles as magnetic concentrators and electrochemical transducers in the detection of nucleic acid targets (e.g., polynucleotides such as DNA which can be characteristic of and enable detection/identification of a particular genus, species, or strain of microorganism). The electrically-active magnetic nanoparticles can be synthesized from gamma iron-oxide templates coated with an electrically-active polymer such as polyaniline. A biosensor detection mechanism encompasses two sets of nucleic acid probes that are specific to the target nucleic acid: detector probes labeled/conjugated to electrically-active magnetic nanoparticles and capture probes labeled with a conjugation or other immobilization moiety such as biotin. Detection can be achieved electrochemically, for example by measuring the oxidation-reduction signal from the conductive polymer of the electrically-active magnetic nanoparticles.

In one aspect, the disclosure relates to a system for hybridizing a target nucleic acid, the system comprising: (a) a biologically enhanced, electrically active magnetic (BEAM) nanoparticle composition comprising: (i) a particulate composition comprising a conductive polymer bound to magnetic nanoparticles; and (ii) a detector probe bound (e.g., covalently bound, ionically bound, via non-specific binding such as adsorption, or via a specific binding interaction) to the conductive polymer of the particulate composition, wherein the detector probe comprises a first oligonucleotide sequence that is complementary to and capable of hybridizing with a first region of the target nucleic acid (e.g., at a first range of base positions in the target nucleic acid); and (b) a capture probe comprising (i) a second oligonucleotide sequence that is complementary to and capable of hybridizing with a second region of the target nucleic acid (e.g., at a second range of base positions in the target nucleic acid) and (ii) a conjugating moiety such as a biotin moiety bound (e.g., covalently bound, ionically bound, via non-specific binding such as adsorption, or via a specific binding interaction) to the second oligonucleotide sequence and capable of specifically binding with a complementary conjugating moiety. More generally, the conjugating moiety of the capture probe can be replaced by any suitable means for immobilization of the capture probe, either alone or hybridized with the target nucleic acid, onto a surface such as a biosensor detector surface.

Various refinements to the disclosed system are possible. In an embodiment, (i) the first oligonucleotide sequence is a linear oligonucleotide having a 5′-end and a 3′-end, and the detector probe is bound to the conductive polymer at either the 5′-end or the 3′-end of the first oligonucleotide sequence; and (ii) the second oligonucleotide sequence is a linear oligonucleotide having a 5′-end and a 3′-end, and the conjugating moiety is bound to the second nucleotide sequence at the 5′-end or the 3′-end of the second oligonucleotide sequence and opposite to the end at which the detector probe is bound to the conductive polymer (e.g., the detector probe is bound to the conductive polymer at the 5′-end of first oligonucleotide sequence and the conjugating moiety is bound to the 3′-end of the second oligonucleotide sequence, or vice versa). In a refinement of the embodiment, (i) the 5′-end of first oligonucleotide sequence is phosphorylated and is covalently bound to the conductive polymer of the particulate composition (e.g., by direct or indirect covalent bonding, such as with an intermediate linker; phosphorylated at 5′-end and covalently linked via phosphoramidite bonds to amino groups of a polyaniline conductive polymer resulting from the reaction between the conductive polymer and the phosphorylated 5′-end with a linker such as EDAC); and (ii) the 3′-end of the second oligonucleotide sequence operably bound to a biotin moiety as the conjugating moiety (e.g., covalently bound to 3′-end, for example via an ester linkage such as between a biotin carboxylic acid group and a terminal hydroxy group at the 3′-end). In a refinement, (i) the first oligonucleotide sequence has from 5 to 100 nucleotide bases; and (ii) the second oligonucleotide sequence has from 5 to 100 nucleotide bases (e.g., at least 5, 10, 15, 20, or 30 and/or up to 10, 20, 30, 40, 60, 80, or 100 bases for either or both, where the two sequences can be independently selected to have different numbers of bases). In another refinement, the first region and the second region of the target nucleic acid are separated by 5 to 100 nucleotide bases (e.g., at least 5, 10, 15, 20, or 30 and/or up to 10, 20, 30, 40, 60, 80, or 100 bases of separation). In another refinement, the detector probe and the capture probe are capable of simultaneously or sequentially hybridizing and specifically binding the target nucleic acid, thereby forming a triplex comprising the detector probe of the BEAM nanoparticle and the capture probe specifically bound to the target nucleic acid (e.g., to the first and second hybridization binding regions thereof, respectively). In another refinement, (i) the magnetic nanoparticles comprise at least one of Fe(II) and Fe(III); and, (ii) the conductive polymer is selected from the group consisting of polyanilines, polypyrroles, polythiophenes, derivatives thereof, combinations thereof, blends thereof with other polymers, and copolymers of the monomers thereof.

In a particular embodiment of the system for hybridizing the target nucleic acid, (i) the first oligonucleotide sequence is 5′-GGAAGAGTGAGGGTGGATACAGGCT-CGAACTGGAGTGAAGTGTTACCGCA-3′ (SEQ ID NO: 2), and the detector probe is covalently bound to the conductive polymer of the particulate composition at the 5′-end of the first oligonucleotide sequence (e.g., phosphorylated at 5′-end and covalently linked via phosphoramidite bonds to amino groups of a polyaniline conductive polymer resulting from the reaction between the conductive polymer and the phosphorylated 5′-end with a linker such as EDAC); (ii) the second oligonucleotide sequence is 5′-GGAAAAGATTTAAAT-CTGGTAGAAAGGCGG-3′ (SEQ ID NO: 3), and the capture probe comprises a biotin moiety operably bound to the capture probe at the 3′-end of the second oligonucleotide sequence as the conjugating moiety (e.g., covalently bound to 3′-end, for example an ester linkage such as between a biotin carboxylic acid group and a terminal hydroxy group at the 3′-end); and (iii) the target nucleic acid comprises a polynucleotide sequence from the protective antigen (pag A) gene of Bacillus anthracis (e.g., the first region represents positions 3289-3338 and the second region represents positions 3373-3402 of the B. anthracis pag A gene as set forth in GENBANK accession number M22589, incorporated herein by reference).

In an embodiment, the system further comprises: (c) a biosensor device comprising the complementary conjugating moiety bound (e.g., covalently bound, ionically bound, via non-specific binding such as adsorption, or via a specific binding interaction) to a zone on the surface of the biosensor device, wherein specific binding between the conjugating moiety of the capture probe and the complementary conjugating moiety of the biosensor device immobilizes the capture probe on the surface of the biosensor device. The biosensor device can be a screen-printed carbon electrode (SPCE) or a membrane strip biosensor, for example a screen-printed carbon electrode (SPCE) where the complementary conjugating moiety is operably bound to a working electrode of the SPCE. In various refinements, (i) the conjugating moiety of the capture probe can be a biotin moiety; (ii) the complementary conjugating moiety of the biosensor device can be selected from the group consisting of streptavidin, avidin, and neutravidin; and/or (iii) the biosensor further comprises gold nanoparticles (AuNP) at the surface to which the complementary conjugating moiety is immobilized.

In another aspect, the disclosure relates to a triplex for detecting a target nucleic acid, the triplex comprising: (a) a biologically enhanced, electrically active magnetic (BEAM) nanoparticle composition comprising: (i) a particulate composition comprising a conductive polymer bound to magnetic nanoparticles; and (ii) a detector probe bound (e.g., covalently bound, ionically bound, via non-specific binding such as adsorption, or via a specific binding interaction) to the conductive polymer of the particulate composition, wherein the detector probe comprises a first oligonucleotide sequence that is complementary to and capable of hybridizing with a first region of a target nucleic acid; (b) a capture probe comprising (i) a second oligonucleotide sequence that is complementary to and capable of hybridizing with a second region of the target nucleic acid and (ii) a conjugating moiety bound (e.g., covalently bound, ionically bound, via non-specific binding such as adsorption, or via a specific binding interaction) to the second oligonucleotide sequence and capable of specifically binding with a complementary conjugating moiety; and (c) the target nucleic acid of the detector probe and the capture probe, wherein the target nucleic acid is hybridized with and specifically bound to both the first oligonucleotide sequence at the first region of the target nucleic acid and the second oligonucleotide sequence at the second region of the target nucleic acid.

In another aspect, the disclosure relates to a method for detecting the presence of a target nucleic acid in a sample, the method comprising: (a) providing a triplex according to any of the various foregoing embodiments for the BEAM nanoparticles, detector probe, capture probe, target nucleic acid, etc.; (b) detecting the triplex; and (c) optionally determining that the target nucleic acid or that an analyte corresponding to the target nucleic acid is present in the sample.

Various refinements to the disclosed method are possible. For example, providing the triplex in part (a) can comprise: (i) forming the triplex in a liquid medium comprising (A) the sample comprising the target nucleic acid, (B) the BEAM nanoparticle composition comprising the detector probe, and (C) the capture probe; and (ii) magnetically separating and concentrating the triplex from the liquid medium (e.g., separating the triplex from other non-target material originally in the sample or otherwise in the liquid medium; more generally forming and separating/concentrating a BEAM-detector probe-target nucleic acid conjugate from a liquid medium, with or without a capture probe bound thereto). Detecting the triplex can comprise (i) acid-doping the conductive polymer of the triplex and then (ii) performing cyclic voltammetry to a biosensor device to which the triplex is immobilized to detect the acid-doped triplex.

In another refinement of the method, providing the triplex in part (a) comprises: (i) contacting the capture probe and the BEAM nanoparticle composition with the sample for a time sufficient to hybridize and specifically bind any target nucleic acid present in the sample to the first oligonucleotide sequence of the detector probe bound to the conductive polymer of the BEAM nanoparticle composition and to the second oligonucleotide sequence of the capture probe, thereby forming the triplex; and (ii) immobilizing the triplex on a surface (e.g., by specific binding between the conjugating moiety of the capture probe in the triplex and a complementary conjugating moiety immobilized on the surface, such as the detection/active surface of a biosensor).

In another refinement of the method, providing the triplex in part (a) comprises: (i) immobilizing the capture probe on a surface (e.g., by specific binding between the conjugating moiety of the capture probe and a complementary conjugating moiety immobilized on the surface, such as the detection/active surface of a biosensor); (ii) contacting the capture probe with the sample for a time sufficient to hybridize and specifically bind any target nucleic acid present in the sample to the second oligonucleotide sequence of the capture probe, thereby forming a capture probe-target nucleic acid conjugate; and (iii) contacting the capture probe-target nucleic acid conjugate with the BEAM nanoparticle composition for a time sufficient to hybridize and specifically bind the detector probe of the BEAM nanoparticle composition to target nucleic acid of the capture probe-target nucleic acid conjugate, thereby forming the triplex immobilized on the surface.

In another refinement of the method, providing the triplex in part (a) comprises: (i) immobilizing the capture probe on a surface (e.g., by specific binding between the conjugating moiety of the capture probe and a complementary conjugating moiety immobilized on the surface, such as the detection/active surface of a biosensor); (ii) contacting the BEAM nanoparticle composition with the sample for a time sufficient to hybridize and specifically bind any target nucleic acid present in the sample to the detector probe of the BEAM nanoparticle composition, thereby forming a target nucleic acid-BEAM nanoparticle conjugate; and (iii) contacting the a target nucleic acid-BEAM nanoparticle conjugate with the capture probe for a time sufficient to hybridize and specifically bind the capture probe to the target nucleic acid-BEAM nanoparticle conjugate, thereby forming the triplex immobilized on the surface.

In another refinement of the method, providing the triplex in part (a) comprises: (i) contacting the capture probe with the sample for a time sufficient to hybridize and specifically bind any target nucleic acid present in the sample to the capture probe, thereby forming a capture probe-target nucleic acid conjugate; (ii) immobilizing the capture probe-target nucleic acid conjugate on a surface (e.g., by specific binding between the conjugating moiety of the capture probe in the conjugate and a complementary conjugating moiety immobilized on the surface, such as the detection/active surface of a biosensor);and (iii) contacting the capture probe-target nucleic acid conjugate with the BEAM nanoparticle composition for a time sufficient to hybridize and specifically bind the detector probe of the BEAM nanoparticle composition to the target nucleic acid of the capture probe-target nucleic acid conjugate, thereby forming the triplex immobilized on the surface.

In another aspect, the disclosure relates to a biodetection method for detecting a target nucleic acid in a sample, the method comprising the steps of: providing hybridization probes for hybridizing to the same target nucleic acid, the hybridization probes comprising an electrically-active magnetic nanoparticle-labeled detector probe and a biotinylated capture probe; hybridizing the target nucleic acid with each of the electrically-active magnetic nanoparticle-labeled detector probe and a biotinylated capture probe in a sample comprising target nucleic acid and non-target nucleic acid; magnetically separating the hybridized target nucleic acid from non-complementary and unreacted nucleic acid; capturing the hybridized target nucleic acid on a substrate through the biotinylated capture probe; and measuring the oxidation-reduction signal of the electrically-active magnetic nanoparticle-labeled detector probe (e.g., by cyclic voltammetry).

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the examples, drawings, and appended claims, with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawings wherein:

FIG. 1A illustrates a biologically enhanced, electrically active magnetic (BEAM) nanoparticle probe and a capture probe for a target nucleic acid along with a triplex formed with the target nucleic acid a method for detecting the triplex.

FIG. 1B illustrates a method for attaching a detector probe to an electrically active magnetic (EAM) nanoparticle to form the BEAM nanoparticle probe.

FIG. 1C illustrates a biosensor for detecting the BEAM nanoparticle probe-target nucleic acid-capture probe triplex.

FIG. 2A is a TEM image of EAM NPs.

FIG. 2B is a graph illustrating the UV-vis absorbance spectra of pure polyaniline and EAM NPs.

FIG. 3 is a graph illustrating the fluorescence signal of pure Ph-PRO probes and unreacted Ph-PRO probes after magnetic separation from EAM NPs (mean fluorescence intensity±SD, n=3; SD=standard deviation, n=no. of replicates).

FIG. 4A is a graph illustrating the cyclic voltammograms (CVs) of EAM NPs in 0.1 M HCl at scan rates of 20, 50, 100, 150 and 200 mV/s.

FIG. 4B is a graph illustrating anodic and cathodic peak current vs. scan rate (mean current±SD, n=3) for the EAM NPs.

FIG. 5 is a graph illustrating the cyclic voltammograms of the bare screen printed electrode, the streptavidin-modified electrode, and EAM-captured target DNA hybrids on the electrode in 0.1 M HCl at 20 mV/s.

FIG. 6A is a graph illustrating the electrochemical response of the biosensor in PCR target concentrations ranging from 0 to 10 ng/μl in 0.1 M HCl at 20 mV/s.

FIG. 6B is a graph illustrating the CV mediated anodic peak current at different target DNA concentrations (mean current±SD, n=3).

While the disclosed compositions, kits, apparatus, and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

The present disclosure relates to electrically-active magnetic (EAM) nanoparticles as concentrators of nucleic acid targets as well as electrochemical transducers for detection of the nucleic acid targets. In a specific example described herein, the target nucleic acid is the Bacillus anthracis protective antigen A (pag A) gene (SEQ ID NO:1). Representative EAM nanoparticles can be synthesized from gamma iron-oxide templates coated with an electrically-active polymer such as polyaniline.

EAM-based biosensor detection according to the disclosure encompasses two sets of nucleic acid probes that are specific to the target gene: a detector probe immobilized on or otherwise bound/labeled to the EAM nanoparticles and a capture probe including a conjugating moiety such as biotin for probe immobilization. The detector probe and capture probe include first and second oligonucleotide sequences that are complementary to and capable of hybridizing with first and second regions of the target nucleic acid, respectively. The nucleic acid targets (for convenience, but not limitation, nucleic acids can be referenced as DNA herein, although it will be understood that the detection system may be adapted to other nucleic acids) are double hybridized to the detector and the capture probes to form an EAM probe-target-capture probe triplex, which can be concentrated from non-specific DNA fragments by applying a magnetic field. Subsequently, the DNA sandwiched targets (EAM detector probe-DNA target-capture probe) are captured on a biosensor surface including a complementary conjugating moiety to the conjugating moiety of the capture probe (e.g., streptavidin modified screen-printed carbon electrodes immobilized through the biotinylated capture probes). Detection can be performed electrochemically by measuring the oxidation-reduction signal of the EAM nanoparticles. Results indicate that such biosensors are able to detect redox signals of the EAM nanoparticles at DNA concentrations at least as low as 0.01 ng/μl.

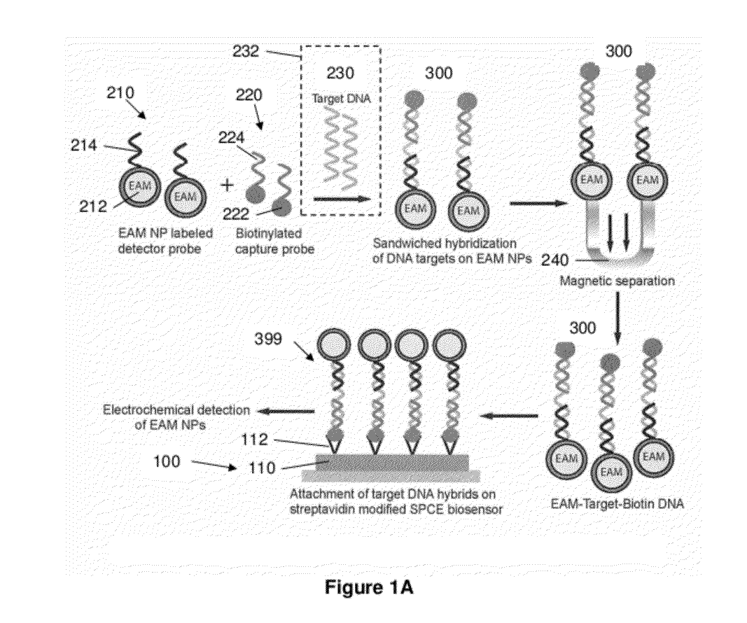

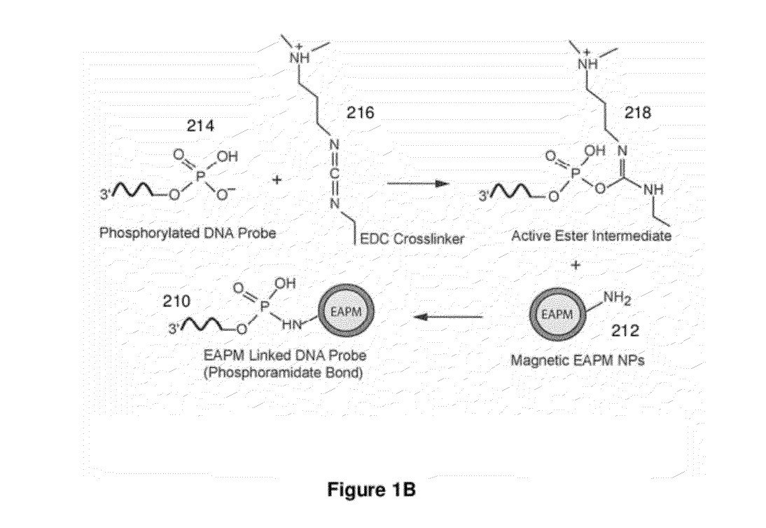

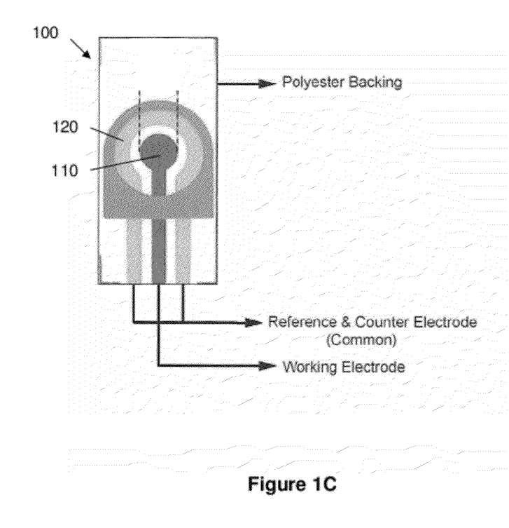

Referring particularly to FIG. 1A, there is shown a schematic representation of the detection mechanism of the EAM-based electrochemical DNA biosensor according to the disclosure. The detection principle involves an electrochemical sandwich assay engaging a detector DNA probe 210 and a capture DNA probe 220. The detector probe 210 includes a first oligonucleotide sequence 214 labeled/conjugated with EAM nanoparticles 212, whereas the capture probe 220 includes a second oligonucleotide sequence 224 labeled/conjugated with a conjugating/immobilization moiety 222 such as biotin. In an embodiment, both the EAM labeled detector probe 210 and the capture probe 220 are combined with a target nucleic acid sequence 230 (e.g., in a sample medium 232 to be tested or other liquid medium), where the target 230 undergoes sandwiched hybridization with both probes 210, 220.

Hybridization refers to the process of establishing a non-covalent, sequence-specific interaction between two or more complementary strands of nucleic acids into a single hybrid (e.g., via pairwise interactions between nucleic bases A=T and GEC), which in the case of two strands is referred to as a duplex. In this case, the detector probe 210 and the capture probe 220 have respective nucleotide sequences 214, 224 that hybridize/specifically bind to different portions/regions of the target DNA sequence 230 (e.g., as represented by non-overlapping hybridization position ranges in the target DNA sequence 230 for the detector probe 210 and the capture probe 220), thus forming a triplex 300 between the detector probe 210, the target nucleic acid 230, and the capture probe 220. The EAM detector probe-target-capture probe hybrids illustrated as the triplexes 300 thus formed are separated from other non-complementary sequences and unreacted DNA by magnetic separation of the unbound EAM nanoparticles 210 and the bound EAM nanoparticles in the triplex 300 (e.g., by magnetic immobilization of the EAM moieties 210, 300 combined with washing of the non-immobilized sample medium and sample components). Subsequently, the EAM detector probe-target-capture probe hybrids 300 are added to the surface of a biosensor 100 (e.g., illustrated as a working electrode 110 of a screen-printed carbon electrode (SPCE) biosensor 100) that is modified with a complementary conjugating moiety 112 (e.g., streptavidin) operably bound/immobilized on the biosensor 100 surface for anchoring the hybrids 300 thereto by specific binding interactions (e.g., streptavidin-biotin interactions). After a short incubation period, the biosensor 100 surface is washed to remove excess EAM nanoparticles 210 (e.g., which have no capture probe 220 and/or target nucleic acid 230 bound thereto) and unbound triplexes 300 (e.g., which may not have had sufficient contact with the complementary conjugating moiety 112 for attachment/immobilization). The DNA targets 230 can be detected on the biosensor 100 surface through the redox properties of the EAM nanoparticles using cyclic voltammetry to detect the presence of the hybrids/triplexes 300 based on the conductive properties of the conductive polymer in the immobilized triplexes 399.

FIG. 1B illustrates suitable cross-linking/covalent attachment chemistry between the EAM nanoparticles 212 and the detector probe 214 using an EDC (1-ethyl-3-[3-dimethylaminopropyl]carbodiimide hydrochloride) covalent linking reagent 216 to form an active ester intermediate 218 between the detector probe 214 and the linking reagent 216, which active ester intermediate 218 reacts with conductive polymer of the EAM nanoparticles 212 (e.g., via amino groups thereon, such as in a polyaniline conductive polymer) to form an EAM-linked detector probe 210.

FIG. 1C illustrates the structure of a screen-printed carbon electrode (SPCE) as a representative biosensor 100 having a reference/counter electrode 120 and a working electrode 110 onto which the hybridized target nucleic acid triplex 300 is immobilized and detected.

The EAM nanoparticle composition can be formed into a biologically enhanced, electrically active magnetic (BEAM) nanoparticle composition by further including a specific binding pair member (e.g., a detector probe in the form of a first oligonucleotide sequence that is complementary to and capable of hybridizing with the target nucleic acid) bound to the conductive polymer of the particulate composition. The BEAM nanoparticle composition can be used as an analyte detection probe in combination with the capture probe. In particular embodiments, the disclosure provides compositions, kits, detection apparatus, and methods for detecting a specific pathogen alone or for detecting a plurality of different particular pathogens in a multiplexed configuration. The disclosed compositions and methods are useful for the rapid, accurate, and selective detection of various target nucleic acids (e.g., which can be characteristic to specific pathogens) using a biosensor platform that is shelf-stable and capable of non-selective binding to multiple analytes in a class encompassing the specific pathogen(s) of interest, such as in assays exploiting the magnetic properties of the nanoparticle compositions (e.g., for analyte concentration) and using any of a variety of detection mechanisms (e.g., conductimetric detection, magnetic detection, using an enzyme label for colorimetric detection).

The BEAM nanoparticle composition can perform a dual function of a magnetic concentrator and a signal transducer in biosensing applications. The magnetic properties of the BEAM nanoparticles serve the purpose of concentrating and separating specific target analytes from complex sample matrices, while the electrical properties of the BEAM nanoparticles can be exploited in various detection schemes, for example biosensing applications which can be based on a conductimetric or other suitable type of assay.

The EAM nanoparticle compositions can mimic the function of magnetic beads widely used as a separator for immunomagnetic separation in immunoassays, for hybridization with nucleic acid probes as capture reagents, as templates in PCR, and the like. In addition, the electrical and the magnetic properties of the nanoparticles or composites can also be exploited as molecular transducers in biosensors. Some of the major advantages of the compositions include: (1) ability to perform the dual function of a magnetic concentrator as well as a biosensor transducer; (2) ability to achieve faster assay kinetics since the compositions are in suspension and in close proximity to target analytes; (3) increased surface area for the biological events to occur; (4) minimized matrix interference due to the improved separation and washing steps; (5) ability to magnetically manipulate the magnetic nanomaterials by using permanent magnets or electromagnets; (6) ability to avoid complex pre-enrichment, purification or pre-treatment steps necessary in standard methods of detection; (7) ability to design cheap, sensitive, highly specific and rapid detection devices for diverse targets by using different biological modifications; and (8) ability to design different rapid detection devices using both electrical and magnetic properties of the BEAM nanoparticles.

### Terms

The term “ssDNA” includes a single free strand of polymerized deoxyribonucleic acids consisting of repeated polymer bases of adenine (A), cytosine (C), guanine (G), and/or thymine (T), where each strand has directionality and runs from five prime (5′) to three prime (3′)

The term “dsDNA” includes a complex of two ssDNA strands that are hybridized to each other in a complimentary fashion (adenine:thymine and cytosine:guanine), the two strands run anti-parallel to each other and form a helical structure, such that at any given end a 5′-end from one strand and a 3′-end from another strand are present.

The term “oligonucleotide” or “strand” includes a DNA molecule having from 2, 4, 6, 8, or 10 bases to 20, 50, 100, 200, 500, or 1000 bases in length and being single stranded.

The term “sequence” includes the specific nucleotide base configuration in a linear 5-prime to 3-prime order.

The term “hybridization” includes the pairing of two oligonucleotides together, where non-covalent bonding occurs between adenine and thymine or cytosine and guanine pairs, and the hybridized oligonucleotides are in opposing orientations during hybridization.

The term “probe” includes the oligonucleotide attached to either EAM nanoparticles or the conjugating moiety and having a sequence selected to hybridize to the target nucleic acid or DNA (e.g., which can be characteristic to a specific organism of interest).

The term “genomic DNA” includes the DNA carried in an organism for normal life-giving functions, where the set of DNA is specific and unique to each organism.

The term “AuNP” includes gold nanoparticles, which can be a solid gold sphere with a diameter of 5 to 50 nanometers.

The term “polymerase chain reaction” (PCR) includes the process of making double stranded copies of either a ssDNA or dsDNA template using an enzymatic assembly process.

The term “complementary” includes a second sequence of DNA bases that mirrors a first sequence, with the second sequence having the following substitutions adenine (A) in place of thymine (T), cytosine (C) in place of guanine (G), thymine (T) in place of adenine (A) and guanine (G) in place of cytosine (C) in an anti-parallel direction relative to the first sequence.

### Electrically-Active Magnetic Particulate Composition

The electrically-active magnetic (EAM) nanoparticle compositions according to the disclosure generally include a conductive polymer bound to magnetic nanoparticles (e.g., a population of magnetic nanoparticles in which each nanoparticle generally has at least some conductive polymer bound thereto, such as a magnetic nanoparticle core with a conductive polymer shell). U.S. Publication No. 2009/0123939, the entire contents of which are hereby incorporated herein by reference, discloses particulate compositions, biologically enhanced particulate compositions and related methods suitable for use according to the present disclosure.

The conductive properties of the conductive polymer (sometimes referenced as a synthetic metal) arise from the π-electron backbone and the single/double bonds of the π-conjugated system alternating down the polymer chain. Some conducting polymeric structures include polyaniline (PANi), polypyrrole, polyacetylene, and polyphenylene. Polyaniline, in particular, has been studied thoroughly because of its stability in fluid form, conductive properties, and strong bio-molecular interactions. Conductive polymers can be used in a biosensor, an analytical device capable of pathogen detection in which the conductive polymers act as electrochemical transducers to transform biological signals to electric signals that can be detected and quantified.

The conductive polymers according to the disclosure are not particularly limited and generally include any polymer that is electrically conductive. Preferably, the conductive polymer is fluid-mobile when bound to an analyte. Suitable examples of conductive polymers are polyanilines, polypyrrole, and polythiophenes, which are dispersible in water and are conductive because of the presence of an anion or cation in the polymer (e.g., resulting from acid-doping of the polymer or monomer). Other electrically conductive polymers include substituted and unsubstituted polyanilines, polyparaphenylenes, polyparaphenylene vinylenes, polythiophenes, polypyrroles, polyfurans, polyselenophenes, polyisothianapthenes, polyphenylene sulfides, polyacetylenes, polypyridyl vinylenes, biomaterials, biopolymers, conductive carbohydrates, conductive polysaccharides, combinations thereof and blends thereof with other polymers, copolymers of the monomers thereof. Conductive polyanilines are preferred. Polyaniline is perhaps the most studied conducting polymer in a family that includes polypyrrole, polyacetylene, and polythiophene. As both electrical conductor and organic compound, polyaniline possesses flexibility, robustness, highly controllable chemical and electrical properties, simple synthesis, low cost, efficient electronic charge transfer, and environmental stability. Addition of a protic solvent such as hydrochloric acid yields a conducting form of polyaniline, with an increase in conductivity of up to ten orders of magnitude. Illustrative are the conductive polymers described in U.S. Pat. Nos. 6,333,425, 6,333,145, 6,331,356 and 6,315,926. Preferably, the conductive polymers do not contain metals in their metallic form.

The conductive polymer provides a substrate for the subsequent attachment of a binding pair member (e.g., a first oligonucleotide sequence as a detector probe) bound thereto, which binding pair member is complementary to a target analyte and thereby forms a BEAM nanoparticle, as described below. The electrically conductive characteristics of the conductive polymer also can facilitate detection of an analyte bound to the BEAM nanoparticle, for example by measuring the electrical resistance or conductance through a plurality of BEAM nanoparticles immobilized in a capture region of conductimetric biosensor device. Additionally, an electrical current passing through plurality of BEAM nanoparticles can be used to induce a magnetic field, and properties such as magnetic permeability or mass magnetization can be detected and correlated to the presence of the target analyte in a sample.

The magnetic nanoparticles according to the disclosure are not particularly limited and generally include any nano-sized particles (e.g., about 1 nm to about 1000 nm) that can be magnetized with an external magnetic/electrical field. The magnetic nanoparticles more particularly include superparamagnetic particles, which particles can be easily magnetized with an external magnetic field (e.g., to facilitate separation or concentration of the particles from the bulk of a sample medium) and then redispersed immediately once the magnet is removed (e.g., in a new (concentrated) sample medium). Thus, the magnetic nanoparticles are generally separable from solution with a conventional magnet. Suitable magnetic nanoparticles are provided as magnetic fluids or ferrofluids, and mainly include nano-sized iron oxide particles (Fe₃O₄ (magnetite) or γ-Fe₂O₃ (maghemite)) suspended in a carrier liquid. Such magnetic nanoparticles can be prepared by superparamagnetic iron oxide by precipitation of ferric and ferrous salts in the presence of sodium hydroxide and subsequent washing with water. A suitable source of γ-Fe₂O₃ is Sigma-Aldrich (St. Louis, Mo.), which is available as a nano-powder having particles sized at <50 nm with a specific surface area ranging from about 50 m²/g to about 250 m²/g. Preferably, the magnetic nanoparticles have a small size distribution (e.g., ranging from about 5 nm to about 25 nm) and uniform surface properties (e.g., about 50 m²/g to about 245 m²/g).

More generally, the magnetic nanoparticles can include ferromagnetic nanoparticles (i.e., iron-containing particles providing electrical conduction or resistance). Suitable ferromagnetic nanoparticles include iron-containing magnetic metal oxides, for example those including iron either as Fe(II), Fe(III), or a mixture of Fe(II)/Fe(III). Non-limiting examples of such oxides include FeO, γ-Fe₂O₃ (maghemite), and Fe₃O₄ (magnetite). The magnetic nanoparticles can also be a mixed metal oxide of the type M1ₓM2₃₋ₓO₄, wherein M1 represents a divalent metal ion and M2 represents a trivalent metal ion. For example, the magnetic nanoparticles may be magnetic ferrites of the formula M1Fe₂O₄, wherein M1 represents a divalent ion selected from Mn, Co, Ni, Cu, Zn, or Ba, pure or in admixture with each other or in admixture with ferrous ions. Other metal oxides include aluminium oxide, chromium oxide, copper oxide, manganese oxide, lead oxide, tin oxide, titanium oxide, zinc oxide and zirconium oxide, and suitable metals include Fe, Cr, Ni or magnetic alloys.

The particulate composition is generally formed by the polymerization of a conductive polymer monomer (e.g., aniline, pyrrole) in a solution (e.g., aqueous) containing the magnetic nanoparticles. The polymerization solution generally includes an acid dopant (e.g., HCl) to impart electrical conductivity to the resulting polymer. The polymerization reaction is preferably initiated by the addition of an oxidant (e.g., ammonium persulfate). Upon completion of the polymerization reaction, the solution contains the particulate composition in which the resulting conductive polymer is bound to the magnetic nanoparticles. The magnetic nanoparticles and the monomer can be combined in any suitable weight ratio in the polymerization solution so that the resulting particulate composition has a desired balance of magnetic, electrical, and particle size properties. For example, the weight ratio of monomer : magnetic nanoparticles in the polymerization solution (or conductive polymer:magnetic nanoparticles in the resulting particulate composition) preferably ranges from about 0.01 to about 10, more preferably from about 0.1 to about 1 or about 0.4 to about 0.8, for example about 0.6. Similarly, the particulate composition preferably ranges in size from about 1 nm to about 500 nm, more preferably about 10 nm to about 200 nm, or about 50 nm or 80 nm to about 100 nm or 120 nm, for example at least about 1 nm, 10 nm, 20 nm, or 50 nm and/or up to about 80 nm, 100 nm, 120 nm, or 200 nm.

### Probes

The detector probe includes a first oligonucleotide sequence that is complementary to and capable of hybridizing with a first region of the target nucleic acid (e.g., at a first range of base positions in the target nucleic acid). Similarly, the capture probe includes second oligonucleotide sequence that is complementary to and capable of hybridizing with a second region of the target nucleic acid (e.g., at a second range of base positions in the target nucleic acid). The capture probe further includes a conjugating moiety that is bound to the second oligonucleotide sequence (e.g., covalently bound, ionically bound, via non-specific binding such as adsorption, or via a specific binding interaction) and is capable of specifically binding with a complementary conjugating moiety.

The EAM nanoparticle composition in any of its above embodiments is extended to a biologically enhanced, electrically active magnetic (BEAM) nanoparticle composition by further including the detector probe as a specific binding pair member to the conductive polymer of the particulate composition. The first oligonucleotide sequence of the detector probe is selected to be complementary to a target nucleic acid so that the BEAM nanoparticle composition can be used for the selective binding and detection of the target nucleic acid in a sample.

The specific binding pair member that is specific to the target analyte can be bound directly or indirectly to the conductive polymer of the particulate composition (or to the detection moiety of the analyte probe more generally) by any of a variety of methods known in the art appropriate for the particular binding pair member (e.g., DNA oligonucleotide). Oligonucleotides can be incubated in a buffer (e.g., an acetate buffer at a pH of about 5.2) suspension of the particulate composition that also includes an immunoconjugating agent (e.g., 1-ethyl-3-(3-dimethylaminopropyl)carbodiimide hydrochloride (“EDAC” “EDC”)). After a suitable incubation period (i.e., depending on the rate of binding between the binding pair member and the conductive polymer) the resulting BEAM nanoparticles can be blocked, washed, centrifuged, and then stored as a suspension (e.g., on a phosphate-buffered saline (“PBS”) solution for an oligonucleotide).

Suitably, the first and second oligonucleotides are linear oligonucleotides bound to their EAM particle or conjugating moiety, respectively, at opposing ends of the their oligonucleotide sequence. For example, the detector probe is bound to the conductive polymer at the 5′-end of first oligonucleotide sequence, and the conjugating moiety is bound to the 3′-end of the second oligonucleotide sequence (or vice versa). As described above, the 5′-end of first oligonucleotide sequence can be phosphorylated and can be covalently bound to the conductive polymer of the particulate composition (e.g., by direct or indirect covalent bonding, such as with an intermediate linker; phosphorylated at 5′-end and covalently linked via phosphoramidite bonds to amino groups of a polyaniline conductive polymer resulting from the reaction between the conductive polymer and the phosphorylated 5′-end with a linker such as EDC). Similarly, the 3′-end of the second oligonucleotide sequence can be covalently bound the conjugating moiety (e.g., via an ester linkage such as between a biotin carboxylic acid group and a terminal hydroxy group at the 3′-end of the second oligonucleotide).

The lengths of the first and second oligonucleotides are not particularly limited, but each may be independently selected to have a suitable length such as from 5 to 100 nucleotide bases (e.g., at least 5, 10, 15, 20, or 30 and/or up to 10, 20, 30, 40, 60, 80, or 100 bases for either or both). Suitably, the first region of the target nucleic acid is selected to be a separate, non-overlapping region relative to the second region of the target nucleic acid (e.g., at least 5, 10, 15, 20, or 30 and/or up to 10, 20, 30, 40, 60, 80, or 100 bases of separation between non-overlapping regions). In an embodiment, the detector probe and the capture probe are capable of simultaneously or sequentially hybridizing and specifically binding the target nucleic acid, thereby forming a triplex between the BEAM-detector probe, the target nucleic acid, and the capture probe.

The conjugating moiety of the capture probe and the complementary conjugating moiety (e.g., as a biosensor component) are suitably specific binding pair members with respect to each other. A specific binding pair member generally includes one of two different molecules, each having a region or area on its surface or in a cavity that specifically binds to (i.e., is complementary with) a particular spatial and polar organization of the other molecule. The binding pair members can be referenced as a ligand/receptor (or antiligand) pair. These binding pair members include members of an immunological pair such as antigen-antibody. Other specific binding pairs such as biotin-avidin (or derivatives thereof such as streptavidin, avidin, or neutravidin), hormones-hormone receptors, IgG-protein A, polynucleotide pairs (e.g., DNA-DNA, DNA-RNA), DNA aptamers, and whole cells are not immunological pairs, but can be used as binding pair members within the context of the present disclosure. Suitably, the binding pair members exhibit a substantial degree of binding specificity to each other and do not exhibit a substantial amount of non-specific binding.

Biotin-avidin/streptavidin pairs are suitable conjugating moiety pairs. Biotin (also known as coenzyme R, vitamin H, or vitamin B7) includes the small molecule C₁₀H₁₆N₂O₃S and derivatives thereof (e.g., ester derivatives between the biotin's carboxylic acid group and a terminal hydroxy group at the 3′-end of the second oligonucleotide) having substantial specific binding affinity to various avidin/streptavidin derivatives. Representative avidin derivatives include the tetrameric protein avidin as well as related derivatives having biotin-binding specificity (e.g., neutravidin as a de-glycosylated form of avidin; recombinant versions of avidin). Representative streptavidin derivatives include the tetrameric protein streptavidin derived from Streptomyces avidinii as well as related derivatives having biotin-binding specificity (e.g., recombinant versions of streptavidin; streptavidin derivatives including the biotin-binding regions of streptavidin).

### Functionalized Biosensor

The detection system for capturing and identifying a target nucleic acid in a sample can include a biosensor device incorporating the complementary conjugating moiety for triplex immobilization. The complementary conjugating moiety can be bound or otherwise immobilized on a surface (e.g., zone or portion thereof) of the biosensor, for example including covalent binding interactions, ionic binding interactions, non-specific binding interactions such as adsorption, or specific binding interactions. As a result, specific binding between the conjugating moiety of the capture probe and the complementary conjugating moiety of the biosensor device immobilizes the capture probe (e.g., alone or in combination with other detection system components such as the target triplex) on the surface of the biosensor device.

FIGS. 1A and 1C illustrates a suitable biosensor 100 (e.g., a screen-printed carbon electrode (SPCE) as shown) for detection of an analyte triplex 300. The illustrated biosensor 100 includes a working electrode 110 as a detection surface and a counter/reference electrode 120. The detection surface 110 includes the complementary conjugating moiety 112 for immobilization of the triplex 300. The detection surface 110 can further include an immobilization means or layer for immobilization of the complementary conjugating moiety 112 (e.g., as a glutaraldehyde layer) and/or other functional biosensor components (e.g., as gold nanoparticles to enhance conductivity in a conductimetric biosensor).

The specific type of biosensor is not particularly limited and generally includes devices known and used for the detection of an analyte or target DNA molecule by combining a biological component (e.g., biological material such as tissue, microorganisms, organelles, cell receptors, enzymes, antibodies, nucleic acids, probe DNA, biomimic, etc.) with a physicochemical transducer element (e.g., an element that works in a physicochemical way; optical, electrical, piezoelectric, electrochemical, etc.) that transforms the signal resulting from the interaction of the analyte with the biological element into another signal (i.e., transducers) that can be measured and quantified. In some embodiments, the transducers act as a means for amplifying a low number or low concentration of analytes in a sample into a detectable and repeatable (meaningful) signal.

As described above, the analyte triplex 300 can be detected once immobilized on an electrode surface of a SPCE biosensor 100. Any suitable biosensor platform may be used, however. For example, a sample containing the triplex 300 can be applied to a capture region of a lateral flow assay device, where the capture region includes the immobilized complementary conjugating moiety 112 (e.g., adsorbed onto a membrane or conjugated/bound thereto). The sample can be applied to the capture region in a variety of ways, such as by direct addition thereto or by capillary transport of the sample from an application region to the capture region. The immobilized complementary conjugating moiety 112 in the capture region retains the analyte triplex 300 in the capture region. The presence of the target analyte in the sample can be determined (e.g., and optionally quantified) by magnetically or conductimetrically detecting the analyte triplex 300 (e.g., by detecting the magnetic nanoparticle or conductive polymer component thereof) in the capture region, inasmuch as EAM detector probes 210 that are not bound to target nucleic acid are transported by capillary action out of the capture region (e.g., into an absorption region of the device).

### Sample

A sample generally includes an aliquot of any matter containing, or suspected of containing, the target analyte/nucleic acid of interest. For example, samples can include biological samples, such as samples from taken from animals (e.g., saliva, whole blood, serum, plasma, urine, tears, milk, and the like), cell cultures, plants; environmental samples (e.g., water); industrial samples; and food samples (e.g., solid or liquid foods in raw or processed form, such as milk). Samples may be required to be prepared prior to analysis according to the disclosed methods. For example, samples may require extraction, dilution, filtration, centrifugation, and/or stabilization prior to analysis. Similarly, samples may be denatured to convert dsDNA from a target organism of interest into corresponding ssDNA strands (e.g., where at least one of which corresponds to the target nucleic acid of interest and is capable of hybridization with the detector probe and capture probe). For the purposes herein, “sample” can refer to either a raw sample as originally collected or a sample resulting from one or more preparation techniques applied to the raw sample. Accordingly, a sample to be tested by contact with an EAM detector probe and/or capture probe can be a liquid medium containing the analyte, where the liquid medium can be the raw sample to be tested (e.g., milk), or it can be a liquid medium (e.g., a buffer) to which a solid or liquid raw sample to be tested is added.

### Target Nucleic Acid Detection

As generally illustrated in FIGS. 1A-1C and described above, the biosensor 100, the detector DNA probe 210, and the capture DNA probe 220 of any of the above embodiments can be used in an assay to detect the presence of the target nucleic acid 230 in the sample 232 (e.g., which can contain the target nucleic acid 230 of interest as well as non-target components, analytes, or nucleic acids). Specific detection of the target nucleic acid 230 is effected using the first oligonucleotide sequence 214 and second oligonucleotide sequence 224 as probe components and selected to be complementary to the target analyte nucleic acid 230. The detector DNA probe 210 and the capture DNA probe 220 can be independently provided in a variety of forms, for example a liquid suspension, a powder, or as part of an assay device (e.g., in an application region of a lateral flow assay device).

The detector DNA probe 210 and the capture DNA probe 220 are contacted with the sample 232 for a time sufficient to bind any target nucleic acid 230 in the sample 232 to the first and second oligonucleotides 214, 224, thereby forming an analyte triplex 300. The sample 232 and probes 210, 220 can be contacted in any convenient way, for example by combining the components in a reaction vessel (e.g., adding the sample 232 to a suspension of the probes 210, 220, adding the probes 210, 220 to a liquid sample 232, adding each component to a third vessel). The contact time required to obtain sufficient binding between the target analyte and the binding pair member generally depends on the kinetics of the particular analyte-binding pair member interaction (e.g., at least 1, 2, 5, or 10 minutes and/or up to 30, 60, 90, or 120 minutes).

A magnetic field can be applied to the sample 232 (e.g., using a magnet 240) to concentrate the analyte triplex 300 using an immunomagnetic separation process. Specifically, the applied magnetic field attracts the magnetic portion of the analyte triplex 300, causing individual particles of the triplex 300 to migrate to and concentrate in a region of the assay reaction vessel. Thus, after migration of the triplex 300, a portion of the sample 232 that is substantially free from the triplex 300 can be removed (e.g., by draining, skimming, pipetting, washing, etc.), thereby forming a sample concentrate that contains substantially all of the triplex 300. The sample concentrate can include some free detector DNA probes 210 that are not conjugated to a target nucleic acid 230 (e.g., resulting from an excess of added DNA probe 210 relative to the target nucleic acid 230 present in the sample 232)

The analyte triplex 300 is then immobilized on the detection surface 110 of the biosensor 100. Immobilization can be effected by contacting the triplex 300 with the detection surface 110 under conditions sufficient to specifically bind the conjugating moiety 222 of the triplex 300 to the complementary conjugating moiety 112 of the biosensor 100. As above, the particular conditions can depend on the specific binding interaction, but room-temperature incubation (e.g., at 20° C. to 30° C.) for 2-60 minutes (e.g., 5-30 minutes, 10-20 minutes) before rinsing and detection is generally suitable. As illustrated in FIGS. 1A and 1C for the specific case of a SPCE biosensor 100, the triplex 300 is immobilized on/over a working electrode 110 (i.e., as the detection surface 110) and adjacent a counter/reference electrode 120 of an electrochemical biosensor 100 for performing conductimetric or electrochemical detection. In this case, the triplex 300 can be applied directly to the detection surface 110 (e.g., by pipetting a liquid suspension of the triplex 300, such as that resulting from an immunomagnetic separation process).

As illustrated and described above, the analyte triplex 300 is formed prior to its immobilization on the detection surface 110. In other embodiments, however, the triplex 300 formation and immobilization can be performed in any desired order. For example, in an embodiment, (i) the capture probe 220 is immobilized on a (biosensor) surface through conjugating moiety 112/222 interactions, (ii) the immobilized capture probe 220 is contacted with the sample 232 to form a capture probe 220-target nucleic acid 230 conjugate with any target nucleic acid 230 present in the sample 232, and (iii) the immobilized capture probe 220-target nucleic acid 230 conjugate is contacted with the DNA detector probe 210 to form the immobilized triplex 399. In another embodiment, (i) the capture probe 220 is immobilized on a (biosensor) surface through conjugating moiety 112/222 interactions, (ii) the DNA detector probe 210 is contacted with the sample 232 to form a DNA detector probe 210-target nucleic acid 230 conjugate with any target nucleic acid 230 present in the sample 232, and (iii) the DNA detector probe 210-target nucleic acid 230 conjugate is contacted with the immobilized capture probe 220 to form the immobilized triplex 399. In another embodiment, (i) the capture probe 220 is contacted with the sample 232 to form a capture probe 220-target nucleic acid 230 conjugate with any target nucleic acid 230 present in the sample 232, (ii) the capture probe 220-target nucleic acid 230 is immobilized on a (biosensor) surface through conjugating moiety 112/222 interactions, and (iii) the immobilized capture probe 220-target nucleic acid 230 conjugate is contacted with the DNA detector probe 210 to form the immobilized triplex 399.

Prior to detection of the analyte triplex 300 (e.g., via the conductive polymer component thereof), the detection surface 110 having the triplex 300 immobilized thereon is suitably washed (e.g., by rinsing with DI water or other suitable wash fluid). Washing enhances the qualitative and quantitative accuracy of the assay, because it provides a means to eliminate free DNA probes 210 on the detection surface 110 that are not bound to any target nucleic acid 230 (but which would otherwise be detectable and contribute to a false positive or positively biased concentration due to the presence of the conductive polymer). Because the free DNA probes 210 are not conjugated to any capture probe 220 (i.e., via the target nucleic acid 230), they are not subject to specific binding/immobilization interactions with the complementary conjugating moiety 112 of the biosensor 100 and they can be freely washed away with the rinse fluid (e.g., along with any other non-target components that may be residually present in the material applied to the detection surface 110)

Similarly prior to detecting the analyte triplex 300, the conductive polymer component thereof is suitably electrically activated. Such re-activation forms an electrically activated analyte triplex 300 having an increased electrical conductivity relative to the triplex 300 as originally formed (e.g., where the electrical conductivity of the conductive polymer as originally formed from a sample can be reduced during sample capture, extraction, and concentration steps). Suitable methods for re-activation include acid-doping the analyte triplex 300, such as by contacting it with a strong acid (e.g., HCl, HNO₃, H₂SO₄) or a weak acid.

The biosensor 100 is then used to detect the presence of the immobilized analyte triplex 300, such as via the conductive polymer component thereof. A positive identification of the triplex 300 in the sample (concentrate) applied to the detection surface 110 indicates the presence of the target nucleic acid 230 in the original sample 232 (e.g., as well as the organism corresponding to the nucleic acid 230). If a quantitative determination of the triplex 300 is made, any dilution and concentration factors can be used to determine the concentration of the target nucleic acid 230 in the original sample 232. The specific method of detection of the analyte-nanoparticle complex is not particularly limiting, and can include methods applicable to immunoassays in general or immunomagnetic assays in particular (e.g., agglomeration, spectrophotometric detection, colorimetric detection, radioactive detection, visual inspection). In the method illustrated in the figures and examples, the electrically conductive polymer component of the EAM nanoparticles and corresponding probe 210 can be conductimetrically or electrochemically detected (e.g., by conducting cyclic voltammetry to detect the conductive polymer moiety of the electrically activated analyte triplex 300).

### Kit

The disclosed compositions also can be provided in a kit. A suitable kit includes one or more of a detector DNA probe 210 specific to a desired nucleic acid 230, a capture DNA probe 220 specific to the desired nucleic acid 230, and a functionalized biosensor 100 with a complementary conjugating moiety 112 capable of specific binding to the conjugating moiety of the capture probe 220. In an embodiment, the kit can be intended to be used for multiplexed analysis of several different target nucleic acids 230 such that it contains a plurality of different probe pairs 210, 220 where each analyte probe pair 210, 220 has specific oligonucleotide sequences 214, 224 capable of specifically binding to a different target nucleic acid 230. In such case, the kit can include one or a plurality of functionalized biosensors 100, in particular where each biosensor has the same complementary conjugating moiety 112, but is nonetheless capable of specific binding to all of the different capture probes 220 (e.g., which all contain the same conjugating moiety 222 regardless of their particular oligonucleotide sequence 224). The kit can generally include a variety of other optional components that may be desired and/or appropriate, for example a reaction vessel (e.g., a container for mixing the probes 210, 220 and a sample 232 to be analyzed), a magnet, wash reagents, detector reagents, positive and/or negative control reagents, assay kit instructions according to any of the methods disclosed herein, and other additives (e.g., stabilizers, buffers). The relative amounts of the various reagents may be varied widely, to provide for concentrations in solution of the reagents that substantially optimize the sensitivity of the assay. Particularly, the reagents may be provided as dry powders (e.g., lyophilized) which on dissolution will provide for a reagent solution having the appropriate concentrations for combining with the sample 232.

### EXAMPLE

The following example illustrates various compositions, apparatus, and methods according to the disclosure for detecting target nucleic acids, but are not intended to limit the scope of the claims appended hereto.

Magnetic polymer nanostructures are a new class of multifunctional nanomaterials that are recently being explored in biosensor devices. This example illustrates the application of electrically active magnetic (EAM) nanoparticles as concentrators of DNA targets as well as electrochemical transducers for detection of the Bacillus anthracis protective antigen A (pag A) gene. The EAM nanoparticles are synthesized by chemical polymerization and have dimensions of 80-100 nm. The biosensor detection encompasses two sets of DNA probes that are specific to the target gene: the detector probe labeled with the EAM nanoparticles and the biotinylated capture probe. The DNA targets are double hybridized to the detector and the capture probes and concentrated from nonspecific DNA fragments by applying a magnetic field. Subsequently, the DNA sandwiched targets (EAM-detector probe—DNA target—capture probe-biotin) are captured on streptavidin modified screen printed carbon electrodes through the biotinylated capture probes. Detection is achieved electrochemically by measuring the oxidation—reduction signal of the EAM nanoparticles. Results indicate that the biosensor is able to detect the redox signal of the EAM nanoparticles at DNA concentrations as low as 0.01 ng/μl.

### Experimental

Chemicals and reagents: Aniline, iron (III) oxide nanopowder, sodium chloride (NaCl), sodium phosphate (monobasic and dibasic), sodium acetate, phosphate buffered saline tablets (0.01 M), formamide, ethylene diamine tetra acetic acid (EDTA), isopropanol, ethanol, sodium dodecyl sulfate (SDS), proteinase K, hydrochloric acid (HCl), Trizma base, ethidium bromide, gel loading solution and streptavidin from Streptomyces avidinii were purchased from Sigma Aldrich (St. Louis, Mo.). ACCUPRIME Taq DNA Polymerase system, ULTRAPURE DNAse RNAse free water and ULTRAPURE agarose were purchased from Invitrogen Corporation (Carlsbad, Calif.). EDC (1-ethyl-3-[3-dimethylam inopropyl]carbodiimide hydrochloride) was obtained from Pierce (Rockford, Ill.).

Selection of B. anthracis DNA primers and probes: The forward and reverse primers for PCR, and the capture and detector probes for biosensor detection were designed and selected from the protective antigen (pag A) gene of B. anthracis (SEQ ID NO: 1). The specificity of the primer and the probe sequences were analyzed using the Basic Local Alignment Search Tool (BLAST). The sequence information of the B. anthracis primer pairs and the probes are given in Table 1 below. The detector probe was phosphorylated at the 5′-end and the capture probe was biotinylated at the 3′-end. All oligonucleotide sequences were purchased from Integrated DNA Technologies, Inc. (Coralville, Iowa).

TABLE 1
Probe and Primer Sequences

| Number | Type | Sequence | Length | Positions |
|---|---|---|---|---|
| SEQ ID | Detector | 5′-ggaagagtga gggtggatac aggctcgaac | 50 | 3289-3338 |
| NO: 2 | probe | tggagtgaag tgttaccgca-3′ | bases | |
| SEQ ID | Capture | 5′-ggaaaagatt taaatctggt | 30 | 3373-3402 |
| NO: 3 | probe | agaaaggcgg-3′ | bases | |
| SEQ ID | Forward | 5′-aaaatggaag agtgagggtg-3′ | 20 | 3284-3303 |
| NO: 4 | primer | | bases | |
| SEQ ID | Reverse | 5′-ccgcctttct accagattta-3′ | 20 | 3383-3402 |
| NO: 5 | primer | | bases | |

B. anthracis culturing, DNA isolation and PCR: B. anthracis Sterne strain obtained from the Michigan Department of Community Health (Lansing, Mich.) was used in this example. The bacterial culture was grown in trypticase soy broth at 37° C. for 24 h and the cells were enumerated by microbial plating in trypticase soy agar (II) plates containing 5% sheep blood (BD Biosciences, MD). Genomic DNA was isolated from 1 ml of a 24 h enrichment culture of B. anthracis following a modified protocol for mammalian DNA extraction. PCR amplification of the isolated target DNA was performed using a DNA thermocycler (Eppendorf, Westbury, N.Y.). The 50 μl PCR mixture consisted of template DNA, forward and reverse primers, PCR buffer [1×], 2′-deoxynucleoside 5′-triphosphate (dNTP) mix [0.2 mM], magnesium chloride (MgCl₂) [1.5 mM], and 1 μl of ACCUPRIME Tag DNA Polymerase. The PCR amplification was run under the following conditions: 95° C. for 2 min; 35 cycles of 95° C. for 30 s, 55° C. for 30 s, 72° C. for 60 s; and 72° C. for 5 min. The amplified product was purified using a PCR purification kit (QIAgen, Valencia, Calif.) and characterized by gel electrophoresis in ethidium bromidestained 2.5% agarose gels (EmbiTec, Calif.). The concentration of the purified PCR DNA was finally determined using a spectrophotometer (NANODROP 1000, ThermoScientific, Wilmington, Del.) and was diluted as needed.

EAM nanoparticle synthesis and characterization: The EAM nanoparticles (NPs) were synthesized by a chemical polymerization process using gamma iron (III) oxide (γ-Fe₂O₃) nanoparticles as a template and coated with the electrically active polyaniline. The γ-Fe₂O₃ nanoparticles were first dispersed in a mixture of 50 ml 1M HCl, 10 ml de-ionized water and 0.4 ml of aniline. This mixture was sonicated for 1 hour in an ice bath in order to disintegrate the agglomerated nanoparticles. This was followed by a slow drop-wise addition of the oxidant (ammonium persulfate) to the above mixture with continuous stirring. The ice bath reaction was continued for an additional 4 h. The final product obtained was filtered, followed by repeated washings with 1M HCl, methanol and diethyl ether and dried at room temperature for 48 hours to obtain a dark green powder. The γ-Fe₂O₃ to the aniline monomer weight ratio was maintained at 1:0.6 in the synthesis procedure.

The magnetic and electrical properties of the NPs were confirmed from Superconducting Quantum Interference Device (SQUID) and Four Point Probe measurements. The structural morphology and size distribution of the NPs were studied using a 200 kV Field Emission Transmission Electron Microscope (JEOL 2200). The presence of the polymer was confirmed by studying the absorbance spectra of the EAMs using a UV-vis-NIR scanning spectrophotometer (UV-3101PC, Shimadzu, Kyoto, Japan). One milliliter of the EAM NP suspension in water was transferred into a quartz cuvette (10 mm path length) and the absorbance was measured by scanning the sample for a wavelength range of 300-1000 nm using a step size of 1 nm.

DNA probe labeling with EAM nanoparticles: The EAM NPs were labeled with the phosphorylated detector DNA probes (Ph-PRO) using phosphoramidate linkage between the amine groups of the polymer and the phosphate group of the oligonucleotides. The NPs were dispersed in 10 mM sodium acetate buffer (pH 5.2) by sonication for 10 min and mixed with the Ph-PRO probes [22.5 μM] and 0.1 M 1-ethyl-3-[3-dimethylaminopropyl]carbodiimide hydrochloride (EDC). The mixture was incubated with shaking in a rotational hybridization oven at room temperature for 4 h (Amerex Instruments Inc., Calif.). The Ph-PRO DNA probe labeled EAM NPs (PRO-EAMs) were then separated by a magnetic separator and washed repeatedly with acetate buffer and DNAse RNAse free water to remove the unbound probes. The conjugation efficiency of the DNA probes with the NPs was determined using 6-carboxy fluorescein (6-FAM) labeled Ph-PRO probes (at 3′-end). Attachment of Ph-PRO detector probes to the EAM NPs was confirmed by fluorescence measurements in a Microplate Fluorometer Reader (VICTOR3, Perkin Elmer, Mass.). Fluorescence of the pure 6-FAM labeled Ph-PRO probe solution and the supernatant solution containing the unreacted probes after magnetic separation of the PRO-EAMs was observed by excitation at 495 nm and detection of emission at 520 nm. The NP concentrations were varied at 0.1, 1, 10 and 20 mg/ml while keeping the Ph-PRO probe concentration fixed. The fluorescence measurements were also confirmed by spectrophotometric UV-vis absorbance measurements at 260 nm.

Dual hybridization and concentration of target DNA: The PCR amplified DNA targets were hybridized dually with the detector probe labeled EAM NPs (PRO-EAMs) and the biotinylated capture probes (PRO-Bio). Before hybridization, the purified PCR product was denatured by heating at 95° C. for 10 min in the thermocycler and cooling in icebath for 5 min, followed by a second cycle of heating at 95° C. for 5 min and cooling for 5 min. One hundred microliters of the denatured PCR product were diluted in 35 μl of hybridization buffer composed of 2 M NaCl and 0.2 M phosphate buffer (pH 7.4) and added to appropriate concentrations of the PRO-EAMs and the PRO-Bio probes; the mixtures were hybridized at 45° C. for 1 h in a rotational hybridization oven. The resulting EAM-target-biotin DNA hybrids were washed twice with tris ethylene diamine tetraacetic acid (TE) buffer (1×, pH 7.4) for 1 min by magnetic separation of the EAM NPs to remove the unbound target DNA. This was followed by washing the EAM NPs with 20% formamide in 0.01 M phosphate buffered saline (PBS) at 50° C. for 1 min by magnetic separation to remove the nonspecifically bound DNA targets. Finally, the concentrated EAM-target-biotin DNA hybrids were resuspended in 40 μl of DNAse RNAse free water.

Optimization of the hybridization conditions was performed in two steps.

The first step involved hybridization of the target DNA with the PRO-EAMs, which was confirmed through fluorescence assays using the Microplate Fluorometer Reader. Fluorescence labeled PCR amplified target DNA was obtained using 5′6-FAM labeled reverse primers in the PCR reaction. The hybridization time and temperature for the target DNA with the PRO-EAMs was determined by observing the supernatant fluorescence after hybridization of the 6-FAM labeled PCR product with PRO-EAMs and magnetic separation by excitation at 495 nm and measurement of emission at 520 nm. Three different hybridization temperatures (25° C., 45° C. and 55° C.) and three different hybridization times 30 minutes, 1 hour and 2 hours) were trialed.

The second step involved confirmation of the sandwiched hybridization of the PRO-Bio probes with the target DNA using the optimized conditions obtained from the above study through fluorescence assays. 5′6-FAM labeled PRO-Bio probes (1 μM, 5 μM and 10 μM) were used to confirm the dual hybridization event by measuring the fluorescence of the pure probe solution and that of the supernatant after magnetic separation.

Sensor design and surface modification: A screen printed three-electrode sensor (Gwent Group, UK) was used in the electrochemical detection of DNA targets. The sensor chip had an overall dimension of 22 mm×12 mm with a circular working electrode of 4 mm diameter and a partially circular (270° C.) common reference and counter electrode of 1.5 mm in width (FIG. 1C). Screen printed carbon acted as working electrode and screen printed silver/silver chloride (Ag/AgCl) acted as the common reference and counter electrode on a polyester backing. The screen-printed carbon and silver ink had a resistance of 50 Ohms at 12 microns, and 320 mOhms at 25 microns, respectively, according to the manufacturer's specifications. The working electrode surface was modified with streptavidin for target binding. Forty microliters of streptavidin solution (1 mg/ml) prepared in 0.1 M phosphate buffer (pH 7.0) were added onto the working electrode surface and incubated for 2 h at room temperature. The electrodes were then dried at ambient temperature for an additional 30 min and were ready for use in biosensor experiments. Approximately, the working electrode surface was coated with 0.8 μg/mm² of streptavidin. For data collection, the biosensor units were connected to a Potentiostat/Galvanostat (Model 263A, PRINCETON APPLIED RESEARCH, Oak Ridge, Tenn.).

Electrochemical detection of EAM nanoparticles and EAM-target-biotin DNA hybrids: Electrochemical detection and characterization of straight EAM NPs were performed on the bare working electrode surface using cyclic voltammetry (CV) in a Potentiostat/Galvanostat (Princeton Applied Research, OakRidge, Tenn.) with two vertex potentials in the ramp mode. One hundred microliters of the NP solution (concentration: 100 μg/ml) in 0.1 M HCl were applied to the electrode surface and allowed to equilibrate for 5 min. The EAM solution was scanned at a scanning potential between −0.4 and +1.0 V and at scan rates ranging from 20 to 200 mV/s.

The EAM-target-biotin DNA hybrids obtained following dual hybridization were detected on streptavidin modified working electrode surface using CV. The concentrated DNA hybrids were allowed to react with the avidin modified electrode surface for 15 min at room temperature and rinsed with DNAse RNAse free water three times to remove any unbound EAM NPs from the surface and dried at ambient temperature for 15 min. One hundred microliters of 0.1 M HCl solution were added to the electrode surface and allowed to equilibrate for 5 min. The CV scans were performed using the same scanning potential window as that of the bare EAMs at a scan rate of 20 mV/s.

Biosensor sensitivity analysis: For sensitivity analysis, the purified PCR products were serially diluted to concentrations ranging from 10⁻¹ to 10⁻⁵ ng/μl using DNAse RNAse free water. Each PCR dilution was then dually hybridized with the PRO-EAM and the PRO-Bio probes and the DNA hybrids were electrochemically detected on the streptavidin modified screen printed electrode surface. A blank control consisting of straight EAM NPs (concentration: 1 mg/ml) suspended in DNAse RNAse free water was tested for comparison. Base line curves for the bare working electrode and the streptavidin modified electrode were also obtained for each experiment.

The CV data obtained as a current vs. potential (I vs. E) curve was used to determine the oxidation (anodic) and reduction (cathodic) peak potentials. The mean and standard deviation of the anodic peak current signal for all target concentrations were estimated on the basis of data from three replicates and the differences between the means were statistically analyzed using single factor analysis of variance (ANOVA).

### Results

Biosensor detection principle: The principle of detection of the EAM based electrochemical DNA biosensor is illustrated in the schematic in FIG. 1A. The detection involves an electrochemical sandwich assay engaging a detector DNA probe and a capture DNA probe. The detector probe is labeled with EAM NPs (PRO-EAM) and the capture probe is labeled with biotin (PRO-Bio). The DNA targets are dually hybridized with the PRO-EAM and the PRO-Bio probes resulting in EAM-target-biotin DNA hybrids. The DNA hybrids are concentrated and separated from other non-complementary sequences and unreacted DNA using a magnetic separation stand. The concentrated DNA target hybrids are then added directly to the surface of streptavidin modified screen printed electrodes for anchoring the hybrids on the electrode surface using streptavidin—biotin interactions. After a brief incubation period, the electrode surface is washed to remove the excess EAM NPs and the unbound DNA hybrids. The target DNA is finally detected electrochemically on the electrode surface exploiting the redox properties of the EAM NPs.

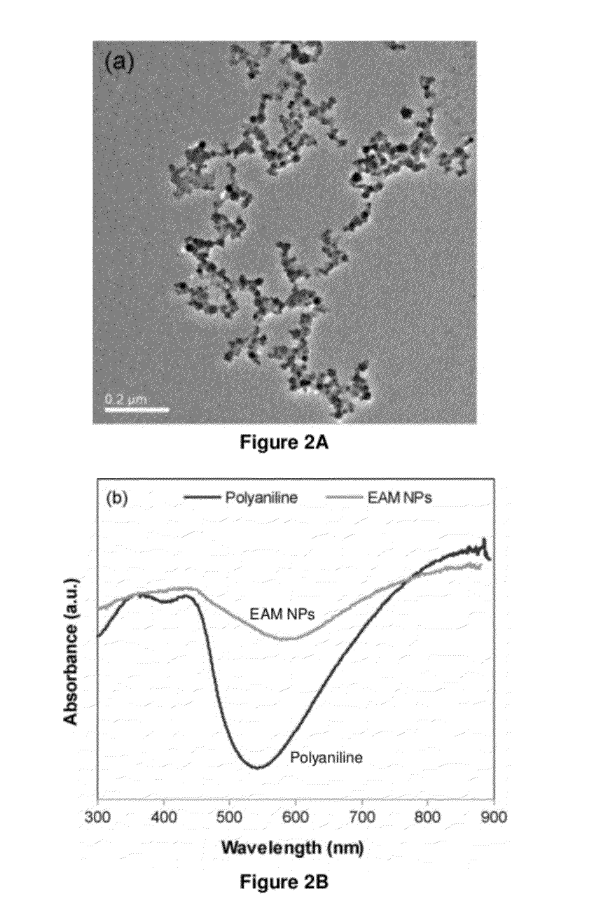

Characterization of EAM nanoparticles: The TEM image of the EAM NPs in FIG. 2A shows that the NPs have an average size of about 80-100 nm. Although some inhomogeneity exists in the NP shape, it is evident from the TEM image that electrically active polyaniline coats the surface of the iron-oxide cores which is essential for the biosensor design as the detection principle is based on the electrochemical properties of the polymer. The presence of the polymer as well as the magnetic core was determined by four point probe and DC SQUID measurements which showed the NPs to have a saturation magnetization value of 44.1 emu/g and an electrical conductivity of 3.3 S/cm. The high electrical conductivity observed in the NPs is imparted by the conducting polymer, polyaniline, in the EAM. The presence of polyaniline in EAM NPs was further confirmed by the UV-vis spectral analysis results in FIG. 2B. As observed in the figure, the characteristic peaks of pure polyaniline appear at 356, 433 and 862 nm. The peak at 356 nm can be attributed to π-π* transition of the benzenoid ring, while the peaks at 433 and 862 nm are associated with polaron-π* and π-polaron band transitions of polyaniline, respectively. For the EAM NPs, characteristic absorption peaks are observed at 441 and 864 nm that can be related to the polaron-π* and π-polaron band transitions of polyaniline. For the EAM NPs, the polaron-π* peak shows an 8 nm shift and the π-polaron peak shows a 2 nm shift from that of pure polyaniline. The red shifts observed in the spectrum can be explained by interactions between the Fe₂O₃ nanoparticles and the polymer backbone. However, the π-π* transition peak of the polymer is not properly distinguished in the absorbance spectrum of the EAM NPs. The absorbance data also confirms that the polymer is present in the doped (conductive) state in the EAM NPs.

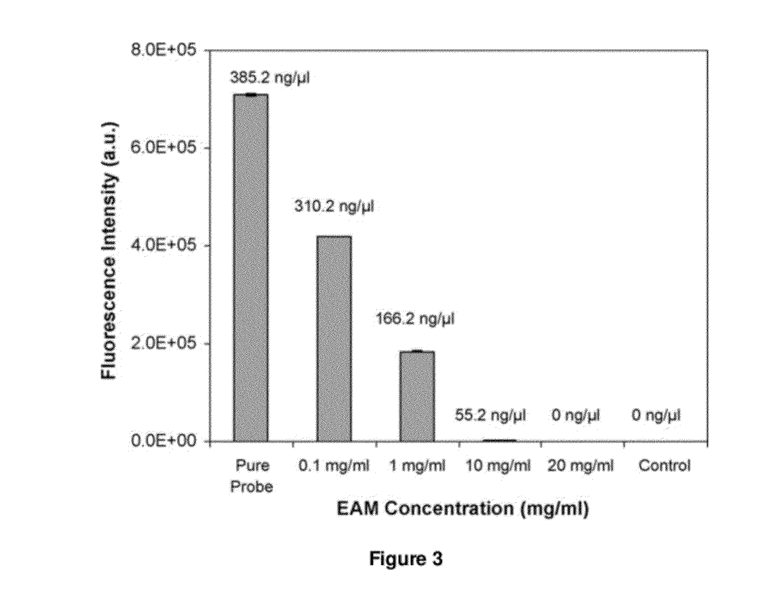

Confirming EAM-DNA probe labeling: The biomodification of the EAM NPs with DNA probes (Ph-PRO) was confirmed by fluorescence and spectrophotometric studies. FIG. 3 shows the fluorescence intensity measurements of the pure 6-FAM labeled Ph-PRO probe solution [22.5 μM] and that of the unreacted probes in the supernatant after magnetic separation of the PRO-EAMs (probe labeled EAMs) at different concentrations of the NPs. As evident in the figure, the 6-FAM labeled Ph-PRO probes (pure probe) show the highest fluorescence signal due to the absence of any EAM NPs in the solution. The supernatants from the labeling process after the magnetic separation of the PRO-EAMs show significantly lower fluorescence signal than that of the pure probe at different EAM concentrations thus indicating the attachment of the probes to the NPs. A linear decrease in the fluorescence signal is also observed as the NP concentration increases from 0.1 to 20 mg/ml which is expected since an increased EAM NP concentration would result in a greater number of attachment sites (terminal amine groups of polyaniline) for the phosphorylated probes. The spectrophotometric measurements of single-stranded DNA (ss-DNA) concentration for the pure probes and the supernatants further confirm the attachment of the probes to the EAM NPs. The ss-DNA concentration for the pure probe is 385.2 ng/μl, whereas for the supernatants from PRO-EAMs, the ss-DNA concentration decreases and is in the range of 310.2-0 ng/μl. A final EAM concentration of 1 mg/ml is chosen for the hybridization of DNA targets since the EAM NPs appear to be saturated with the DNA probes at this concentration.

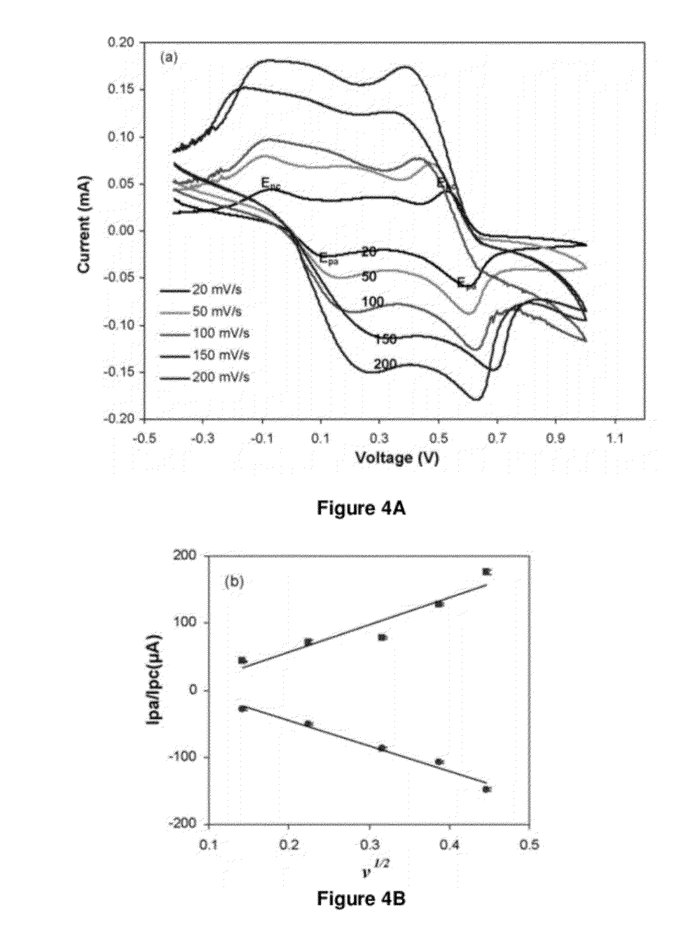

Electrochemical characterization of the EAM nanoparticles: Electrochemical characterization of the synthesized EAM NPs was performed on the working electrodes using CV before proceeding to the detection of EAM-target-biotin DNA hybrids. The electrochemical property of the conducting polymer (polyaniline) in the EAM NPs is strongly dependent on doping the polymer with the ions present in the electrolyte and therefore its redox-activity is prevalent in the protonated (doped) state that occurs at around pH<4. For this reason, 0.1 M HCl was chosen as the electrolyte in all CV scans of the EAM NPs to ensure the electrically active state of the polymer in the NPs. Additionally, the potential window (−0.4 to +1.0 V) chosen for the CV experiments was favorable for the redox process of the polymer in the NPs over that of the γ-Fe₂O₃ core which is electrochemically reduced at potentials lower than −0.4 V. FIG. 4A shows the cyclic voltammograms of the EAM NPs in 0.1 M HCl scanned from −0.4 to +1.0 V at scan rates ranging from 20 to 200 mV/s. As evident in the figure, two well-defined redox peaks (the anodic and cathodic peaks are indicated as Eₚₐ and E_{pc} in the figure) of the EAM NPs are visible at the different scan rates. The two redox peaks obtained can be attributed to the characteristic electrochemical behavior of polyaniline. The broad anodic peak at +0.118 V (at 20 mV/s scan rate) corresponds to the switching of the leucoemeraldine base form to the emeraldine salt form of polyaniline in the EAM NPs. The sharper anodic peak at +0.600 V (at 20 mV/s scan rate) is conventionally attributed to the oxidation of the emeraldine form to the pernigraniline salt form of the polymer. The corresponding cathodic peaks for the reduction process are observed at +0.530 and −0.070 V, respectively. The effect of scan rate on the rate of electron transfer to the electrode surface by the EAM NPs is also evident in FIG. 4A. With an increase in the scan rate, the anodic peak potential of the EAM NPs is shifted to a more positive potential, the peak current is increased, and the cathodic peak potential is shifted to a negative potential. FIG. 4B shows a plot of the dependence of the anodic peak current and the cathodic peak current vs. the scan rate. As observed in the figure, the peak current exhibits a linear relationship with increasing scan rate thus suggesting surface controlled behavior and diffusion controlled system. However, the ratio of the peak currents at different scan rates Iₚₐ/I_{pc}≠1 which indicates a quasi-reversible chemistry of the EAM NPs and the electrode process.

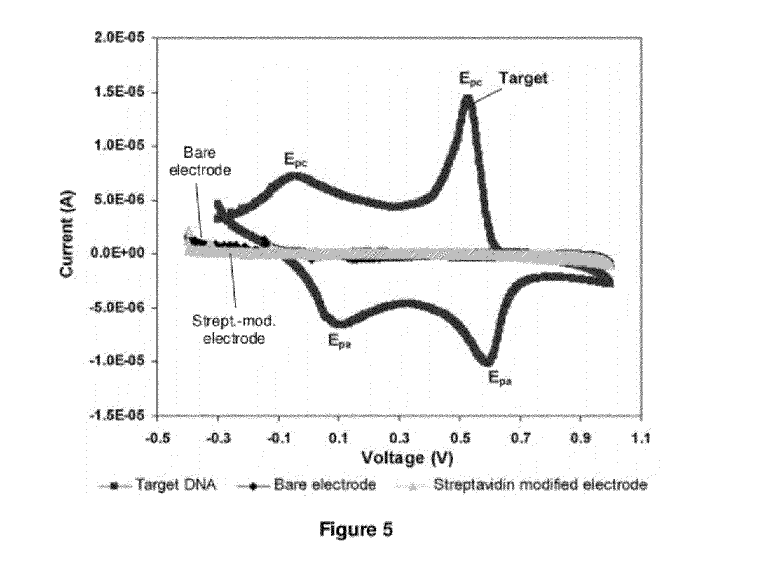

Detection of B. anthracis DNA: The DNA hybridization conditions were determined from fluorescence assays as described above. The optimal hybridization time and temperature for dual hybridization of the DNA targets with the capture and detector probes as determined from supernatant fluorescence measurement was 30 min at 45° C. FIG. 5 shows the electrochemical response of the concentrated EAM-target-biotin DNA hybrids from undiluted PCR products (7.3 ng/μl) captured on streptavidin modified working electrode following dual hybridization. The electrochemical responses of the bare electrode and streptavidin modified electrode were compared as control. As evident, the CV of the EAM captured targets shows the two characteristic redox peaks of the EAM NPs that are absent in the control. The anodic peak potentials are located at +0.110 and +0.597 V, whereas the cathodic peak potentials are located at +0.530 and −0.070 V, similar to observations with bare EAM NPs in 0.1 M HCl. The presence of the redox peaks in the CV response demonstrates that the EAM NPs are electrochemically active after the probe labeling and sandwiched hybridization processes and also confirms the dual function of the EAM NPs, i.e. successful magnetic capture of target DNA and electrochemical detection of the concentrated EAM-target-biotin DNA hybrids.

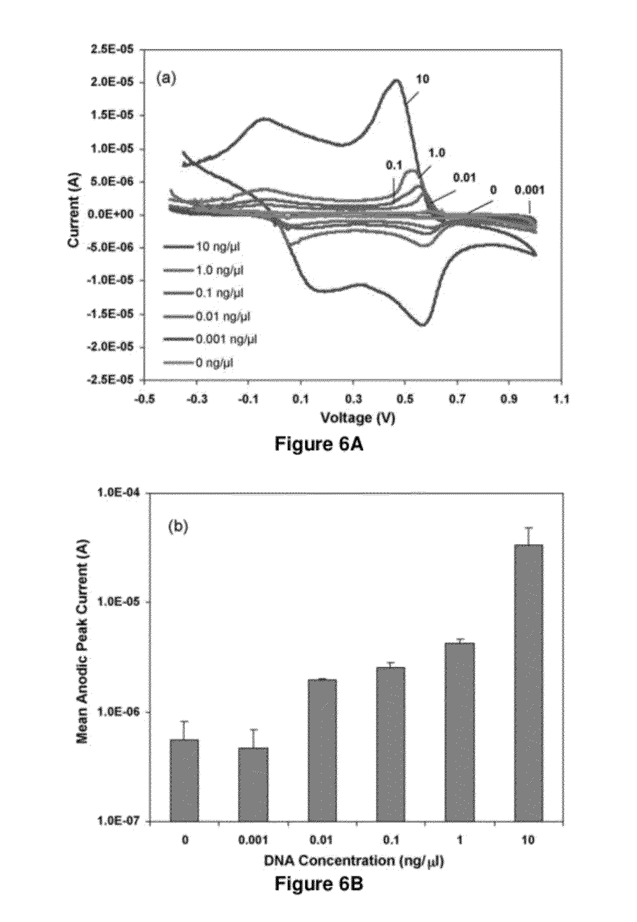

Sensitivity analysis of B. anthracis DNA targets: FIG. 6A shows the electrochemical response of the biosensor in PCR target concentrations ranging from 0.001 to 10 ng/μl and the control. The control solution consisted of probe labeled EAM NPs hybridized with 0 ng/μl of the PCR target solution. As observed in the CV response, the two characteristic redox peaks of the EAM NPs are present at different concentrations of the PCR target. A gradual decrease in the intensity of the redox peaks is noted with a decrease in the target concentration. The anodic peak current at +0.59V is maximum (17 μA) for the highest PCR target concentration (10 ng/μl) and decreases to 1.95 μA at a target concentration of 0.01 ng/μl which is expected as a low target concentration would imply fewer EAM-target DNA-biotin hybrids bound to the electrode surface. The anodic peak currents observed for the control and DNA concentration of 0.001 ng/μl are 0.84 and 0.29 μA, respectively. The presence of EAM redox peaks in the CV scans also confirms the ability of the EAM NPs to capture and detect DNA targets from low DNA concentrations.

Preliminary sensitivity analysis of the detected B. anthracis DNA targets was performed from the CV results. Although both redox peaks of EAM NPs were evident at lower target concentrations in FIG. 6A, the anodic peak current (oxidation peak) at the peak potential of +0.59V was chosen for the sensitivity analysis due to minimal shifts observed in the peak current values in repeated observations. FIG. 6B shows the mean anodic peak current values observed for the different target concentrations from three experimental replicates. A hundredfold increase in the anodic current signal is observed as the target DNA concentration increases from 0 to 10 ng/μl. At a potential of 0.59 V, the mean anodic peak current value from three experimental trials is 33.6×10⁻⁶ (±14.07×10⁻⁶)A for the highest PCR target concentration (10 ng/μl) and decreases to 1.99×10⁻⁶ (±0.03×10⁻⁶)A at the target concentration of 0.01 ng/μl. The mean anodic peak currents for the control and the lowest tested concentration of 0.001 ng/μl are 0.55×10⁻⁶ (±0.27×10⁻⁶)A and 0.47×10⁻⁶ (±0.21×10⁻⁶)A, respectively. The slightly higher current values observed for the control as compared to 0.001 ng/μl of target implies no detection at this concentration and can be attributed to signal from non-specifically adsorbed EAM NPs on the electrode surface in both cases.

The sensitivity result obtained from the CV response was statistically analyzed by one-way single factor analysis of variance (ANOVA) of the anodic peak current signal for the different DNA concentrations at a significance of 95% (P<0.05) using the SAS software. The lowest DNA concentration that showed an anodic peak current signal significantly different (P<0.05) from the control was considered as the detection limit of the sensor. The statistical results indicated that the different target DNA concentrations had a significant effect (P<0.0001) on the anodic peak current signal. The anodic peak current for DNA concentrations ranging from 10 to 0.01 ng/μl were significantly different from control with P value<0.0001, whereas for DNA concentration of 0.001 ng/μl the P value was 0.6943. Therefore, the lower limit of detection of the EAM based DNA sensor was determined to be 0.01 ng/μl of DNA from the ANOVA results. Furthermore, the signal-to-noise ratio (S/N) of the mean anodic peak current at 0.01 ng/μl DNA concentration and the control is greater than 3 (S/N=1.99×10⁻⁶/0.55×10⁻⁶). Additionally, the ANOVA of the current signal between different DNA target concentrations (10-0.01 ng/μl) was significantly different (P≦0.05) from each other indicating the feasibility of quantitative estimation of target DNA using the EAM based biosensor.

### Summary

This example illustrates the use of EAM nanoparticles as a magnetic concentrator of DNA targets and a nanostructured transducer in the electrochemical detection of DNA. The representative EAM based DNA biosensor is fabricated and implemented in the detection of B. anthracis pag A gene. The detection platform was also found to have a high sensitivity: the sensitivity of the biosensor as determined from cyclic voltammetry measurements is 0.01 ng/μl of DNA in a total detection time of 60 min.

Because the EAM nanoparticles are used for both sample concentration and detection, the present invention enables simpler, faster, and more efficient sample processing and detection from complex sample matrices. The EAM nanoparticles have faster assay kinetics, since the nanoparticles are in suspension and are proximal to the target molecules. The EAM nanoparticles also possess a high surface-to-volume ratio, which enables more opportunities for biological activity.

Further, the versatile detection system enables a platform for other biosensing applications, for example being useful for detecting pathogens in a variety of sample types and settings, such as homeland security, defense, water quality monitoring, food processing, and clinical diagnostics.

Because other modifications and changes varied to fit particular operating requirements and environments will be apparent to those skilled in the art, the disclosure is not considered limited to the examples chosen for purposes of illustration, and covers all changes and modifications which do not constitute departures from the true spirit and scope of this disclosure.

Accordingly, the foregoing description is given for clarity of understanding only, and no unnecessary limitations should be understood therefrom, as modifications within the scope of the disclosure may be apparent to those having ordinary skill in the art.

Throughout the specification, where the compositions, kits, processes, or apparatus are described as including components, steps, or materials, it is contemplated that the compositions, kits, processes, or apparatus can also comprise, consist essentially of, or consist of, any combination of the recited components or materials, unless described otherwise. Component concentrations expressed as a percent are weight-percent (% w/w), unless otherwise noted. Numerical values and ranges can represent the value/range as stated or an approximate value/range (e.g., modified by the term “about”). Combinations of components are contemplated to include homogeneous and/or heterogeneous mixtures, as would be understood by a person of ordinary skill in the art in view of the foregoing disclosure.

What is claimed is:

1. A system for hybridizing a target nucleic acid, the system comprising:
(a) a biologically enhanced, electrically active magnetic (BEAM) nanoparticle composition comprising: (i) a particulate composition comprising a conductive polymer bound to magnetic nanoparticles; and (ii) a detector probe bound to the conductive polymer of the particulate composition, wherein the detector probe comprises a first oligonucleotide sequence that is complementary to and capable of hybridizing with a first region of the target nucleic acid; and
(b) a capture probe comprising (i) a second oligonucleotide sequence that is complementary to and capable of hybridizing with a second region of the target nucleic acid and (ii) a conjugating moiety bound to the second oligonucleotide sequence and capable of specifically binding with a complementary conjugating moiety.

2. The system of claim 1, wherein:
(i) the first oligonucleotide sequence is a linear oligonucleotide having a 5′-end and a 3′-end, and the detector probe is bound to the conductive polymer at either the 5′-end or the 3′-end of the first oligonucleotide sequence; and
(ii) the second oligonucleotide sequence is a linear oligonucleotide having a 5′-end and a 3′-end, and the conjugating moiety is bound to the second nucleotide sequence at the 5′-end or the 3′-end of the second oligonucleotide sequence and opposite to the end at which the detector probe is bound to the conductive polymer.

3. The system of claim 2, wherein:
(i) the 5′-end of first oligonucleotide sequence is phosphorylated and is covalently bound to the conductive polymer of the particulate composition; and
(ii) the 3′-end of the second oligonucleotide sequence operably bound to a biotin moiety as the conjugating moiety.

4. The system of claim 1, wherein:
(i) the first oligonucleotide sequence has from 5 to 100 nucleotide bases; and
(ii) the second oligonucleotide sequence has from 5 to 100 nucleotide bases.

5. The system of claim 1, wherein the first region of the target nucleic acid is a separate, non-overlapping region relative to the second region of the target nucleic acid.

6. The system of claim 5, wherein the first region and the second region of the target nucleic acid are separated by 5 to 100 nucleotide bases.

7. The system of claim 1, wherein:
(i) the first oligonucleotide sequence is 5′-GGAAGAGTGAGGGTGGATACAGGCT-CGAACTGGAGTGAAGTGTTACCGCA-3′ (SEQ ID NO: 2), and the detector probe is covalently bound to the conductive polymer of the particulate composition at the 5′-end of the first oligonucleotide sequence;
(ii) the second oligonucleotide sequence is 5′-GGAAAAGATTTAAATCTGGTAGAAA-GGCGG-3′ (SEQ ID NO: 3), and the capture probe comprises a biotin moiety operably bound to the capture probe at the 3′-end of the second oligonucleotide sequence as the conjugating moiety; and
(iii) the target nucleic acid comprises a polynucleotide sequence from the protective antigen (pag A) gene of Bacillus anthracis.

8. The system of claim 1, wherein the conjugating moiety of the capture probe is a biotin moiety.

9. The system of claim 1, further comprising:
(c) a biosensor device comprising the complementary conjugating moiety operably bound to a zone on the surface of the biosensor device, wherein specific binding between the conjugating moiety of the capture probe and the complementary conjugating moiety of the biosensor device immobilizes the capture probe on the surface of the biosensor device.

10. The system of claim 9, wherein the biosensor device is a screen-printed carbon electrode (SPCE) or a membrane strip biosensor.

11. The system of claim 9, wherein the biosensor device is a screen-printed carbon electrode (SPCE), and the complementary conjugating moiety is operably bound to a working electrode of the SPCE.

12. The system of claim 9, wherein:
(i) the conjugating moiety of the capture probe is a biotin moiety; and
(ii) the complementary conjugating moiety of the biosensor device is selected from the group consisting of streptavidin, avidin, and neutravidin.

13. The system of claim 9, further comprising gold nanoparticles (AuNP) at the surface to which the complementary conjugating moiety is immobilized.

14. The system of claim 1, wherein the detector probe and the capture probe are capable of simultaneously or sequentially hybridizing and specifically binding the target nucleic acid, thereby forming a triplex comprising the detector probe of the BEAM nanoparticle and the capture probe specifically bound to the target nucleic acid.

15. The system of claim 1, wherein:
(i) the magnetic nanoparticles comprise at least one of Fe(II) and Fe(III); and, (ii) the conductive polymer is selected from the group consisting of polyanilines, polypyrroles, polythiophenes, derivatives thereof, combinations thereof, blends thereof with other polymers, and copolymers of the monomers thereof.

16. A triplex for detecting a target nucleic acid, the triplex comprising:
(a) a biologically enhanced, electrically active magnetic (BEAM) nanoparticle composition comprising: (i) a particulate composition comprising a conductive polymer bound to magnetic nanoparticles; and (ii) a detector probe bound to the conductive polymer of the particulate composition, wherein the detector probe comprises a first oligonucleotide sequence that is complementary to and capable of hybridizing with a first region of the target nucleic acid;
(b) a capture probe comprising (i) a second oligonucleotide sequence that is complementary to and capable of hybridizing with a second region of the target nucleic acid and (ii) a conjugating moiety bound to the second oligonucleotide sequence and capable of specifically binding with a complementary conjugating moiety; and
(c) the target nucleic acid of the detector probe and the capture probe, wherein the target nucleic acid is hybridized with and specifically bound to both the first oligonucleotide sequence at the first region of the target nucleic acid and the second oligonucleotide sequence at the second region of the target nucleic acid.

17. A method for detecting the presence of a target nucleic acid in a sample, the method comprising:
(a) providing the triplex of claim 16; and
(b) detecting the triplex.

18. The method of claim 17, wherein providing the triplex in part (a) comprises:
(i) immobilizing the triplex on a surface of a biosensor device comprising the complementary conjugating moiety operably bound to the surface of the biosensor device, wherein specific binding between the conjugating moiety of the capture probe and the complementary conjugating moiety of the biosensor device immobilizes the triplex.

19. The method of claim 18, wherein:
(i) the conjugating moiety of the capture probe is a biotin moiety; and
(ii) the complementary conjugating moiety of the biosensor device is selected from the group consisting of streptavidin, avidin, and neutravidin.

20. The method of claim 17, wherein providing the triplex in part (a) comprises:
(i) forming the triplex in a liquid medium comprising (A) the sample comprising the target nucleic acid, (B) the BEAM nanoparticle composition comprising the detector probe, and (C) the capture probe; and
(ii) magnetically separating and concentrating the triplex from the liquid medium.

21. The method of claim 17, wherein providing the triplex in part (a) comprises:
(i) contacting the capture probe and the BEAM nanoparticle composition with the sample for a time sufficient to hybridize and specifically bind any target nucleic acid present in the sample to the first oligonucleotide sequence of the detector probe bound to the conductive polymer of the BEAM nanoparticle composition and to the second oligonucleotide sequence of the capture probe, thereby forming the triplex; and
(ii) immobilizing the triplex on a surface.

22. The method of claim 17, wherein providing the triplex in part (a) comprises:
(i) immobilizing the capture probe on a surface;
(ii) contacting the capture probe with the sample for a time sufficient to hybridize and specifically bind any target nucleic acid present in the sample to the second oligonucleotide sequence of the capture probe, thereby forming a capture probe-target nucleic acid conjugate; and
(iii) contacting the capture probe-target nucleic acid conjugate with the BEAM nanoparticle composition for a time sufficient to hybridize and specifically bind the detector probe of the BEAM nanoparticle composition to target nucleic acid of the capture probe-target nucleic acid conjugate, thereby forming the triplex immobilized on the surface.

23. The method of claim 17, wherein providing the triplex in part (a) comprises:
(i) immobilizing the capture probe on a surface;
(ii) contacting the BEAM nanoparticle composition with the sample for a time sufficient to hybridize and specifically bind any target nucleic acid present in the sample to the detector probe of the BEAM nanoparticle composition, thereby forming a target nucleic acid-BEAM nanoparticle conjugate; and
(iii) contacting the a target nucleic acid-BEAM nanoparticle conjugate with the capture probe for a time sufficient to hybridize and specifically bind the capture probe to the target nucleic acid-BEAM nanoparticle conjugate, thereby forming the triplex immobilized on the surface.

24. The method of claim 17, wherein providing the triplex in part (a) comprises:
(i) contacting the capture probe with the sample for a time sufficient to hybridize and specifically bind any target nucleic acid present in the sample to the capture probe, thereby forming a capture probe-target nucleic acid conjugate;
(ii) immobilizing the capture probe-target nucleic acid conjugate on a surface; and
(iii) contacting the capture probe-target nucleic acid conjugate with the BEAM nanoparticle composition for a time sufficient to hybridize and specifically bind the detector probe of the BEAM nanoparticle composition to the target nucleic acid of the capture probe-target nucleic acid conjugate, thereby forming the triplex immobilized on the surface.

25. The method of claim 17, wherein detecting the triplex comprises (i) acid-doping the conductive polymer of the triplex and then (ii) performing cyclic voltammetry to a biosensor device to which the triplex is immobilized to detect the acid-doped triplex.

26. The method of claim 17, further comprising determining that the target nucleic acid or that an analyte corresponding to the target nucleic acid is present in the sample.


SEQUENCE LISTING (5 sequences)

SEQ ID NO: 1 - DNA, 4235 bp, Bacillus anthracis <s300> M22589.1 1999-08-02
aagcttctgtcattcgtaaatttcaaatagaacgtaaatttagacttctcatcattaaaaatgaaaaatcttatctttttgattctattgtatatttttattaaggtgtttaatagttagaaaagacagttgatgctattactccagataaaatatagctaaccataaatttattaaagaaaccttgttgttctaaataatgattttgtggattccggaatagatactggtgagttagctctaattttatagtgatttaactaacaatttataaagcagcataattcaaattttttaattgatttttcctgaagcatagtataaaagagtcaaggtcttctagacttgactcttggaatcattaggaattaacaatatatataatgcgctagacagaatcaaattaaatgcaaaaatgaatattttagtaagagatccatatcattatgataataacggtaatattgtaggggttgatgattcatatttaaaaaacgcatataagcaaatacttaattggtcaagcgatggagtttctttaaatctagatgaagatgtaaatcaagcactatctggatatatgcttcaaataaaaaaaccttcaaaccacctaacaaacagcccagttacaattacattagcaggcaaggacagtggtgttggagaattgtatagagtattatcagatggagcaggattcctggatttcaataagtttgatgaaaattggcgatcattagtagatcctggtgatgatgtttatgtgtatgctgttactaaagaagattttaatgcagttactcgagatgaaaatggtaatatagcgaataaattaaaaaacaccttagttttatcgggtaaaataaaagaaataaacataaaaactacaaatattaatatatttgtagtttttatgtttattatatacctcctattttatattattagtagcacagtttttgcaaatcatgtaattgtatacttatctatgtagaggtatcacaacttatgaatagtgtattttattgaacgttggttagcttggacagttgtatggatatgcatactttataacgtataaaatttcacgcaccacaataaaactaatttaacaaaaacaaaaacacacctaagatcattcagttcttttaataaggagctgcccaccaagctaaacctaaataatctttgtttcacataaggtttttttctaaatatacagtgtaagttattgtgaatttaaccagtatatattaaaaatgttttatgttaacaaattaaattgtaaaacccctcttaagcatagttaagaggggtaggttttaaattttttgttgaaattagaaaaaataataaaaaaacaaacctattttctttcaggttgtttttgggttacaaaacaaaaagaaaacatgtttcaaggtacaataattatggttctttagctttctgtaaaacagccttaatagttggatttatgactattaaagttagtatacagcatacacaatctattgaaggatatttataatgcaattccctaaaaatagttttgtataaccagttcttttatccgaactgatacacgtattttagcataatttttaatgtatcttcaaaaacagcttctgtgtccttttctattaaacatataaattcttttttatgttatatatttataaaagttctgtttaaaaagccaaaaataaataattatctctttttatttatattatattgaaactaaagtttattaatttcaatataatataaatttaattttatacaaaaaggagaacgtatatgaaaaaacgaaaagtgttaataccattaatggcattgtctacgatattagtttcaagcacaggtaatttagaggtgattcaggcagaagttaaacaggagaaccggttattaaatgaatcagaatcaagttcccaggggttactaggatactattttagtgatttgaattttcaagcacccatggtggttacctcttctactacaggggatttatctattcctagttctgagttagaaaatattccatcggaaaaccaatattttcaatctgctatttggtcaggatttatcaaagttaagaagagtgatgaatatacatttgctacttccgctgataatcatgtaacaatgtgggtagatgaccaagaagtgattaataaagcttctaattctaacaaaatcagattagaaaaaggaagattatatcaaataaaaattcaatatcaacgagaaaatcctactgaaaaaggattggatttcaagttgtactggaccgattctcaaaataaaaaagaagtgatttctagtgataacttacaattgccagaattaaaacaaaaatcttcgaactcaagaaaaaagcgaagtacaagtgctggacctacggttccagaccgtgacaatgatggaatccctgattcattagaggtagaaggatatacggttgatgtcaaaaataaaagaacttttctttcaccatggatttctaatattcatgaaaagaaaggattaaccaaatataaatcatctcctgaaaaatggagcacggcttctgatccgtacagtgatttcgaaaaggttacaggacggattgataagaatgtatcaccagaggcaagacacccccttgtggcagcttatccgattgtacatgtagatatggagaatattattctctcaaaaaatgaggatcaatccacacagaatactgatagtgaaacgagaacaataagtaaaaatacttctacaagtaggacacatactagtgaagtacatggaaatgcagaagtgcatgcgtcgttctttgatattggtgggagtgtatctgcaggatttagtaattcgaattcaagtacggtcgcaattgatcattcactatctctagcaggggaaagaacttgggctgaaacaatgggtttaaataccgctgatacagcaagattaaatgccaatattagatatgtaaatactgggacggctccaatctacaacgtgttaccaacgacttcgttagtgttaggaaaaaatcaaacactcgcgacaattaaagctaaggaaaaccaattaagtcaaatacttgcacctaataattattatccttctaaaaacttggcgccaatcgcattaaatgcacaagacgatttcagttctactccaattacaatgaattacaatcaatttcttgagttagaaaaaacgaaacaattaagattagatacggatcaagtatatgggaatatagcaacatacaattttgaaaatggaagagtgagggtggatacaggctcgaactggagtgaagtgttaccgcaaattcaagaaacaactgcacgtatcatttttaatggaaaagatttaaatctggtagaaaggcggatagcggcggttaatcctagtgatccattagaaacgactaaaccggatatgacattaaaagaagcccttaaaatagcatttggatttaacgaaccgaatggaaacttacaatatcaagggaaagacataaccgaatttgattttaatttcgatcaacaaacatctcaaaatatcaagaatcagttagcggaattaaacgcaactaacatatatactgtattagataaaatcaaattaaatgcaaaaatgaatattttaataagagataaacgttttcattatgatagaaataacatagcagttggggcggatgagtcagtagttaaggaggctcatagagaagtaattaattcgtcaacagagggattattgttaaatattgataaggatataagaaaaatattatcaggttatattgtagaaattgaagatactgaagggcttaaagaagttataaatgacagatatgatatgttgaatatttctagtttacggcaagatggaaaaacatttatagattttaaaaaatataatgataaattaccgttatatataagtaatcccaattataaggtaaatgtatatgctgttactaaagaaaacactattattaatcctagtgagaatggggatactagtaccaacgggatcaagaaaattttaatcttttctaaaaaaggctatgagataggataaggtaattctaggtgatttttaaattatctaaaaaacagtaaaattaaaacatactctttttgtaagaaatacaaggagagtatgttttaaacagtaatctaaatcatcataatcctttgagattgtttgtaggatcc

SEQ ID NO: 2 - DNA, 50 bp, Artificial Sequence
  Synthetic oligonucleotide
ggaagagtgagggtggatacaggctcgaactggagtgaagtgttaccgca

SEQ ID NO: 3 - DNA, 30 bp, Artificial Sequence
  Synthetic oligonucleotide
ggaaaagatttaaatctggtagaaaggcgg

SEQ ID NO: 4 - DNA, 20 bp, Artificial Sequence
  Synthetic oligonucleotide
aaaatggaagagtgagggtg

SEQ ID NO: 5 - DNA, 20 bp, Artificial Sequence
  Synthetic oligonucleotide
ccgcctttctaccagattta